(19) United States
(12) Patent Application Publication
(10) Pub. No.: US 2015/0374718 A1
(43) Pub. Date: Dec. 31, 2015

(54) Zhankuic Acid A and Analogs thereof and Their Use as an Anti-Inflammatory Agent

(71) Applicant: National Cheng Kung University, Tainan (TW)

(72) Inventors: Tian-Shung WU, Tainan (TW); Chao-Liang WU, Tainan (TW); Ai-Li SHIAU, Tainan (TW); Yu-Fon CHEN, Tainan (TW)

(73) Assignee: NATIONAL CHENG KUNG UNIVERSITY, Tainan (TW)

(21) Appl. No.: 14/314,741

(22) Filed: Jun. 25, 2014

(51) Int. Cl. A61K 31/575; C07J 71/00; C07J 9/00

(52) U.S. Cl. CPC A61K 31/575; C07J 9/00; C07J 71/0005

(57) ABSTRACT

Zhankuic acid A (ZAA) is the major pharmacologically active compound of Taiwanofungus camphoratus. We analyzed the structure of human TLR4/MD-2 complex with ZAA by X-score and HotLig modeling approaches. Two antibodies against MD-2 were used to verify the MD-2/ZAA interaction. The inflammation and survival of the mice pretreated with ZAA and injected with LPS were monitored. The modeling structure shows that ZAA binds the MD-2 hydrophobic pocket exclusively via specific molecular recognition; the contact interface is dominated by hydrophobic interactions. Binding of ZAA to MD-2 reduced antibody recognition to native MD-2, similar to the effect of LPS binding. Furthermore, ZAA significantly ameliorated LPS-induced endotoxemia and Salmonella-induced diarrhea in mice. Our results indicate that ZAA, which can compete with LPS for binding to MD-2 as a TLR4/MD-2 antagonist, is a potential therapeutic agent for Gram-negative bacterial infections.

### FIELD OF THE INVENTION

The present invention is related to a novel us of Zhankuic acid A and analogs thereof as an Anti-Inflammatory Agent.

### BACKGROUND OF THE INVENTION

Lipopolysaccharide (LPS) is a glycolipid endotoxin composed of the amphipathic component lipid A, a hydrophilic polysaccharide core, and an O-antigen outermost domain. LPS generally exists in the outer membrane of various gram-negative bacteria (1). The mammalian immune system recognizes LPS as a foreign molecule, which is the first step toward alerting the host to the possibility of an invasive gram-negative bacterial infection. In mammals, CD14 and the TLR_{4/}MD-2 complex participate in the cellular recognition of LPS (2, 3). Binding of the TLR_{4/}MD-2 complex to LPS triggers the TLR_{4/}MAPK signaling pathway, which includes p38, ERK, and JNK (4). LPS also induces an inflammatory response in resting cells through the phosphorylation and subsequent degradation of IκBα, promoting the nuclear translocation of NF-κB and the NF-κB-stimulated expression of inflammatory genes, such as inducible nitric oxide synthase (iNOS), TNF-α, and IL-6 (5, 6). Thus, high levels of LPS in the circulation may lead to severe sepsis, a life-threatening inflammatory syndrome. However, effective treatments for LPS-mediated inflammatory conditions are not yet available.

Taiwanofungus camphoratus (stout camphor fungus) is a parasitic fungus that only grows on the inner heartwood wall of Cinnamomum kanehirai. This fungus has been widely used in Chinese medicine to treat drug intoxication, diarrhea, abdominal pain, hypertension, and cancer (7). The methanol extracts of T. camphoratus exhibit anti-inflammatory activity in microglia cells through inhibition of iNOS and cyclooxygenase-2 (COX2) expression (8). ZAA, the predominant pharmacologically active compound in the fruiting body of T camphoratus, has been shown to prevent inflammatory responses by human neutrophils, without exerting significant cytotoxicity (9). Moreover, ZAA also displays potent anti-inflammatory activity by inhibiting LPS-induced NO production (10). However, the mechanisms of regulations and ameliorations of inflammation by ZAA have not been well elucidated.

### SUMMARY OF THE INVENTION

In this invention, we investigated the ability of ZAA to reduce the inflammation resulting from gram-negative bacterial infections through the blockade of LPS actions in a mouse model of S. choleraesuis-induced diarrhea. Using X-score and HotLig modeling approaches (11, 12), we show that ZAA could act as a ligand for MD-2, thereby suppressing the LPS/MD-2 interaction. Moreover, ZAA inhibited the NF-κB signaling pathway and reduced TNF-α and IL-6 levels in vitro and in vivo. Intraperitoneal administration of ZAA protected mice against LPS-induced lung and renal injury and S. choleraesuis-induced diarrhea. A series of ZAA analogs were also investigated and found having anti-inflammatory activity in LPS-induced TNF-α expression.

This invention suggests that ZAA and a series of ZAA analogs can potentially act as a therapeutic agent to protect against inflammatory diseases caused by Gram-negative bacterial infections.

According to one aspect of the present invention, the present invention provides a method of treating an inflammatory disease in a subject comprising administering to the subject a compound having the following chemical formula (I) or a pharmaceutically acceptable salt thereof in need of said treatment:

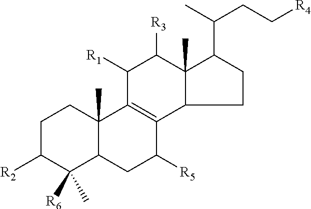

wherein R₁ is ═O; R₂ is ═O, OCHO, or OH; R₃ is H or OH; R₄ is —C(═CH₂)—C(CH₃)H—(C═O)ORₐ, in which Rₐ is H or C₁₋₄ alkyl, or R₄ is —(C═O)R_{b}, in which R_{b} is C₁₋₄ alkyl; R₅ is ═O, OH or H; and R₆ is H or OH.

According to another aspect of the present invention, the present invention provides a use of the compound having the formula (I) or a pharmaceutically acceptable salt thereof as an active ingredient in the fabrication of a medicament for treating an inflammatory disease in a subject.

Preferably, the inflammatory disease is a LPS-mediated inflammatory condition.

Preferably, the inflammatory disease is caused by bacterial infection, and more preferably a Gram-negative bacterial infection.

Preferably, the inflammatory disease comprises LPS-induced lung injury or LPS-induced renal injury.

Preferably, the inflammatory disease comprises diarrhea.

Preferably, the inflammatory disease comprises enteritis.

Preferably, the compound having the formula (I) is selected from the group consisting of

| No. | Compound | Structure |
|---|---|---|
| 1 | Zhankuic acid A | 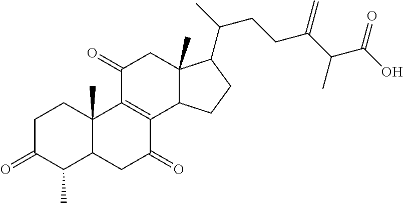 [SMILES: C=C(CCC(C)C1CCC2C3=C(C(=O)C[C@@]21C)[C@@]1(C)CCC(=O)[C@@H](C)C1CC3=O)C(C)C(=O)O] |
| 2 | Zhankuic acid A methyl ester | 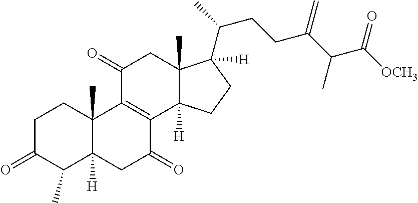 [SMILES: C=C(CC[C@@H](C)[C@H]1CC[C@H]2C3=C(C(=O)C[C@]12C)[C@@]1(C)CCC(=O)[C@@H](C)[C@@H]1CC3=O)C(C)C(=O)OC] |
| 3 | Zhankuic acid B | 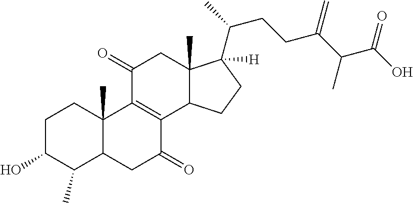 [SMILES: C=C(CC[C@@H](C)[C@H]1CCC2C3=C(C(=O)C[C@@]21C)[C@@]1(C)CC[C@@H](O)[C@@H](C)C1CC3=O)C(C)C(=O)O] |
| 4 | Zhankuic acid C | 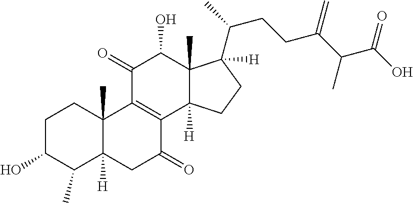 [SMILES: C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)C(C)C(=O)O] |
| 5 | Zhankuic acid C 3-O-formate | 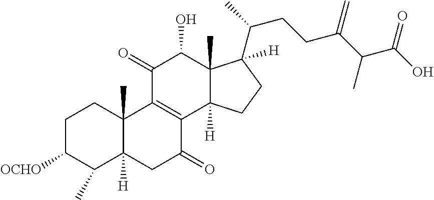 |
| 6 | Zhankuic acid D | 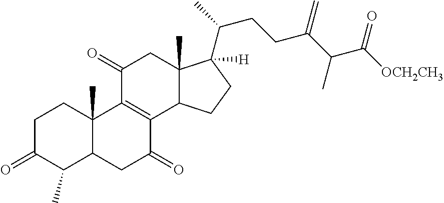 [SMILES: C=C(CC[C@@H](C)[C@H]1CCC2C3=C(C(=O)C[C@@]21C)[C@@]1(C)CCC(=O)[C@@H](C)C1CC3=O)C(C)C(=O)OCC] |
| 7 | Antcin A | 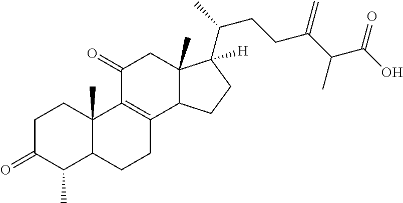 [SMILES: C=C(CC[C@@H](C)[C@H]1CCC2C3=C(C(=O)C[C@@]21C)[C@@]1(C)CCC(=O)[C@@H](C)C1CC3)C(C)C(=O)O] |
| 8 | Antcin A methyl ester | 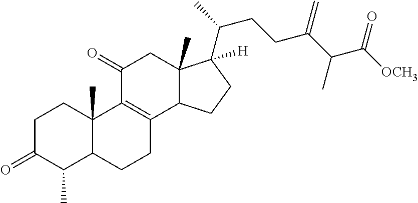 [SMILES: C=C(CC[C@@H](C)[C@H]1CCC2C3=C(C(=O)C[C@@]21C)[C@@]1(C)CCC(=O)[C@@H](C)C1CC3)C(C)C(=O)OC] |
| 9 | Antcin C | 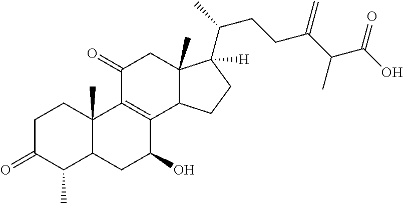 [SMILES: C=C(CC[C@@H](C)[C@H]1CCC2C3=C(C(=O)C[C@@]21C)[C@@]1(C)CCC(=O)[C@@H](C)C1C[C@@H]3O)C(C)C(=O)O] |
| 10 | Antcin K | 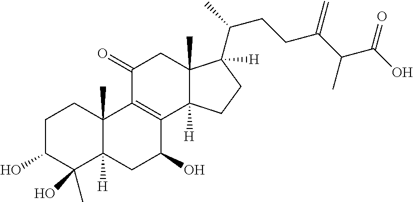 [SMILES: C=C(CC[C@@H](C)[C@H]1CC[C@H]2C3=C(C(=O)C[C@]12C)[C@@]1(C)CC[C@@H](O)[C@](C)(O)[C@@H]1C[C@@H]3O)C(C)C(=O)O] |
| 11 | Antcin M 3-O-formate | 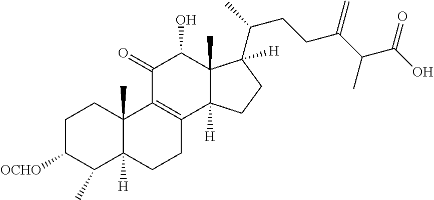 |
| 12 | Camphoratin A | 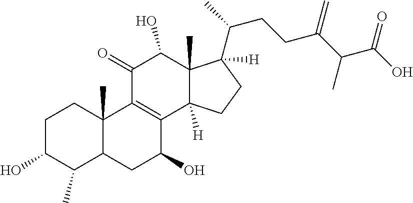 [SMILES: C=C(CC[C@@H](C)[C@H]1CC[C@H]2C3=C(C(=O)[C@H](O)[C@]12C)[C@@]1(C)CC[C@@H](O)[C@@H](C)C1C[C@@H]3O)C(C)C(=O)O] |
| 13 | Camphoratin B | 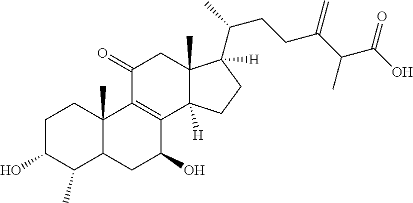 [SMILES: C=C(CC[C@@H](C)[C@H]1CC[C@H]2C3=C(C(=O)C[C@]12C)[C@@]1(C)CC[C@@H](O)[C@@H](C)C1C[C@@H]3O)C(C)C(=O)O] |
| 14 | Camphoratin D | 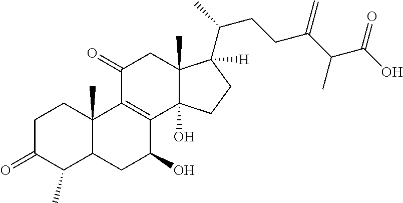 [SMILES: C=C(CC[C@@H](C)[C@H]1CC[C@@]2(O)C3=C(C(=O)C[C@]12C)[C@@]1(C)CCC(=O)[C@@H](C)C1C[C@@H]3O)C(C)C(=O)O] |
| 15 | Camphoratin E | 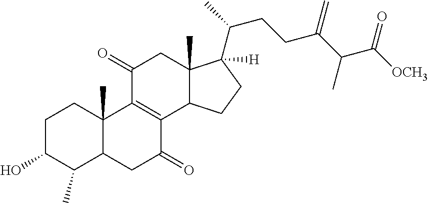 [SMILES: C=C(CC[C@@H](C)[C@H]1CCC2C3=C(C(=O)C[C@@]21C)[C@@]1(C)CC[C@@H](O)[C@@H](C)C1CC3=O)C(C)C(=O)OC] |
| 16 | Camphoratin F | 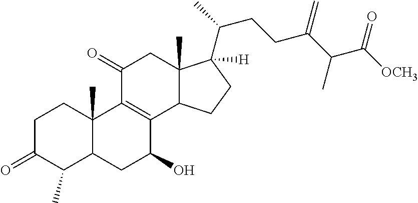 [SMILES: C=C(CC[C@@H](C)[C@H]1CCC2C3=C(C(=O)C[C@@]21C)[C@@]1(C)CCC(=O)[C@@H](C)C1C[C@@H]3O)C(C)C(=O)OC] |
| 17 | Camphoratin G | 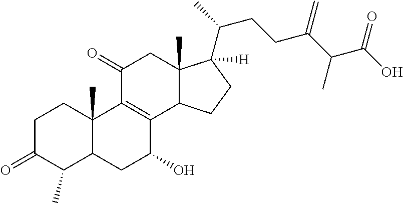 [SMILES: C=C(CC[C@@H](C)[C@H]1CCC2C3=C(C(=O)C[C@@]21C)[C@@]1(C)CCC(=O)[C@@H](C)C1C[C@H]3O)C(C)C(=O)O] |
| 19 | Camphoratin J | 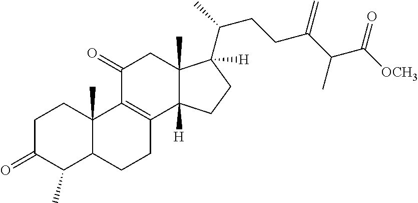 [SMILES: C=C(CC[C@@H](C)[C@H]1CC[C@@H]2C3=C(C(=O)C[C@@]21C)[C@@]1(C)CCC(=O)[C@@H](C)C1CC3)C(C)C(=O)OC] |
| 21 | Camphoratin L | 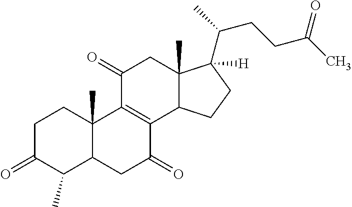 [SMILES: CC(=O)CC[C@@H](C)[C@H]1CCC2C3=C(C(=O)C[C@@]21C)[C@@]1(C)CCC(=O)[C@@H](C)C1CC3=O] |
| 22 | Camphoratin N | 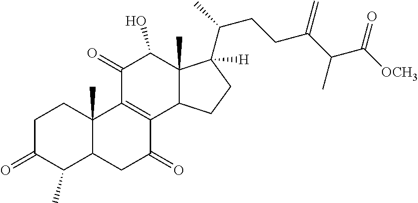 [SMILES: C=C(CC[C@@H](C)[C@H]1CCC2C3=C(C(=O)[C@H](O)[C@@]21C)[C@@]1(C)CCC(=O)[C@@H](C)C1CC3=O)C(C)C(=O)OC] |
| 25 | Ergosterol peroxide | 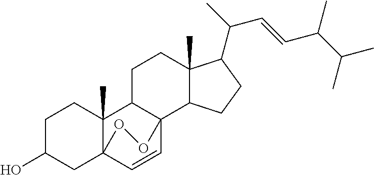 [SMILES: CC(C)C(C)/C=C/C(C)C1CCC2C34C=CC5(CC(O)CC[C@]5(C)C3CC[C@]12C)OO4] |
| 26 | Methyl-4α-methylergost- 8,24(28)-diene-3,11-dion-26- oate | 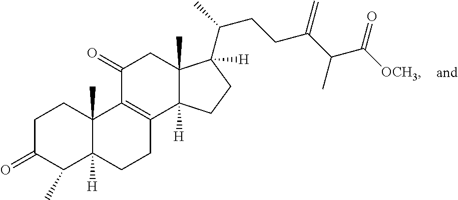 [SMILES: C=C(CC[C@@H](C)[C@H]1CC[C@H]2C3=C(C(=O)C[C@]12C)[C@@]1(C)CCC(=O)[C@@H](C)[C@@H]1CC3)C(C)C(=O)OC ; "and"] |
| 27 | Methyl antcinate H | 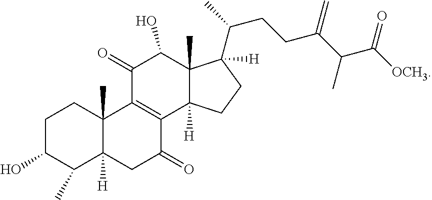 [SMILES: C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)C(C)C(=O)OC] |

### BRIEF DESCRIPTION OF THE DRAWINGS

FIG. 1. ZAA inhibits the production of inflammation-related molecules in LPS- and IFN-γ-stimulated murine macrophages. (A) Murine peritoneal macrophages were pretreated with or without ZAA for 1 h, followed by incubation with LPS (0.5 μg/mL) for 24 h. Total cell lysates were subjected to immunoblotting for detecting COX2 and iNOS. Relative expression levels of COX2 and iNOS protein were quantified by densitometric analysis with ImageJ software and normalized according to the β-actin reference band. (B) Murine peritoneal macrophages were pretreated with ZAA for 1 h, followed by incubation with LPS (0.5 μg/mL) or IFN-γ (50 ng/mL) for 24 h. The presence of nitrite in the culture medium was analyzed by the Griess assay and used as an indication of NO levels (n=4; *p<0.05 and ***p<0.001 vs. LPS- or IFN-γ-induced cells). (C) Raw264.7 cells were cotransfected with pNFKB-Luc and pβ-actin-LacZ plasmids. After 48 h, the cells were treated with or without ZAA for 1 h and then treated with LPS (0.5 μg/mL) for 24 h. Total cell lysates were harvested, and their luciferase activities were determined and normalized on the basis of β-galactosidase activities. Values are means±SD (n=4; *p<0.05 and **p<0.01 vs. LPS-stimulated cells). (D and E) ZAA inhibits NF-κB, MAPK, and Akt signaling pathways in LPS-stimulated RAW264.7 cells. Cells were treated with ZAA for 1 h, followed by stimulation with LPS (0.5 μg/mL) for 30 min. Total cell lysates were examined for the indicated proteins by immunoblotting. Numbers below the blots in D and those shown in the table below the blots in E represent the relative expression levels quantified by densitometric analysis with ImageJ software and normalized according to the β-actin reference bands. Similar results were obtained in three independent experiments. N.D., not detectable.

FIG. 2. ZAA interacts with the hydrophobic binding pocket of MD-2. (A) ZAA assumes a matched configuration to fit into the binding pocket otherwise occupied by LPS. The clipped surface model of MD-2 (PDB entry: 3FXI) is represented along with the ribbon model to depict the LPS binding pocket buried inside the MD-2 protein. The amino acids G1y110-Asn158 shown in magenta, which reside within the immunogenic peptide fragment of MD-2 amino acids 110-160, are around the ZAA-binding site in the ribbon model of MD-2. Gly110 and Asn158 are indicated by arrows. (B) Interactions involved in the binding of ZAA to the hydrophobic amino acid residues within the MD-2 binding pocket are shown. (C) The docked ZAA conformer (depicted in black) is shown superimposed over the terminal carbon chains of LPS within the complex structure of the 3FXI MD-2 model. (D) Recombinant human MD-2 protein (0.15 μg) was incubated with LPS (1 or 10 μg) or ZAA for 3 h, subjected to native PAGE, and immunoblotted with antibodies against different antigenic determinants of MD-2 (amino acids 110-160 and 2-160). (E) Recombinant human TLR_{4/}MD-2 complex (1 μg) was incubated with LPS (10 μg) or ZAA for 3 h, subjected to native PAGE, and immunoblotted with an antibody against MD-2 (amino acids 110-160). Similar results were obtained in three independent experiments.

FIG. 3. ZAA inhibits TNF-α and IL-6 production in LPS- or S. choleraesuis-treated RAW264.7 cells and mice. (A and C) RAW264.7 cells in 96-well plate (2×10⁴ cells/well) were incubated with ZAA for 1 h, followed by treatment with LPS (0.25 or 0.5 μg/mL) (A) or S. choleraesuis (2×10³ CFU/well) (C). Their supernatants collected after 4 and 6 h were assessed for TNF-α and IL-6 levels by ELISA, respectively. (B) C3H/HeJ and C3H/HeN mice were pretreated intraperitoneally with 2 mg/kg of ZAA for 30 min, followed by intraperitoneal injection of LPS (4 mg/kg). (D) C57BL/6 mice were pretreated with 10 mg/kg of ZAA for 30 min, followed by oral administration of S. choleraesuis (2×10⁹ CFU/mouse). Levels of TNF-α and IL-6 were measured in the plasma after 6 h by ELISA. Values are means±SD (n=6-8; *p<0.05, **p<0.01, and ***p<0.001) Similar results were obtained in at least three independent experiments. S.C., S. choleraesuis.

FIG. 4. ZAA reduces LPS-induced pathological changes in mice. (A-D) C3H/HeN and C3H/HeJ mice were pretreated intraperitoneally with ZAA (20 mg/kg) or the vehicle for 30 min and then injected intraperitoneally with LPS (4 mg/kg). After 10 h, mice were sacrificed, and their lung and kidney tissues were removed. (A) Representative microscopic images of hematoxylin-and-eosin-stained sections of lung and kidney tissues are shown. (B) The number of infiltrated PMNs in each alveolus was observed by light microscopy (original magnification ×200). The number of PMNs was counted in four randomly chosen fields per slide for each mouse and normalized to the number of alveoli. (C and D) ZAA decreases the levels of BUN (C) and serum creatinine (D). Values shown in B-D are means ±SD (n=10; **p<0.01 and ***p<0.001). (E) C3H/HeN and C3H/HeJ mice that had been pretreated intraperitoneally with ZAA (2 or 10 mg/kg) or the vehicle for 30 min were injected intraperitoneally with a lethal dose of LPS (20 mg/kg). Survival time was monitored and Kaplan-Meier survival curves were shown in four groups (n=10; ***p<0.001 vs. LPS-treated C3H/HeN mice) Similar results were obtained in at least three independent experiments.

FIG. 5. ZAA ameliorates S. choleraesuis-induced diarrhea, body weight loss, and infection in the gastrointestinal tract. (A and B) C57BL/6 mice that had been pretreated intraperitoneally with ZAA (2 or 10 mg/kg) or the vehicle for 30 min were orally administered with kanamycin-resistant S. choleraesuis (2×10⁹ CFU/mouse). Diarrhea was scored after 2 days on a 0-3 scale (0=normal pellets, 1=slightly loose feces, 2=loose feces, and 3=watery diarrhea) (A). Body weight was recorded every 2 days for 2 weeks (n=9-12; **p<0.01 and ***p<0.001) (B). (C) Fecal samples were collected at 24-h intervals until 96 h after S. choleraesuis infection and assessed for viable bacterial CFU counts (n=10; **p<0.01 and ***p<0.001). (D and E) C57BL/6 mice that had been orally treated with ZAA (2 mg/kg) or the vehicle for 30 min were orally administered with pCMV-Luc-transformed S. choleraesuis (2×10⁸ CFU/mouse). After 48 h, bioluminescence imaging of the mice was conducted after injection with D-luciferin. Whole body images are shown in D. The photon flux scale is shown on the right. (E) Quantification of bioluminescent imaging data. Radiance values are expressed as means±SD (***p<0.001). S.C., S. choleraesuis.

FIG. 6. ZAA is not cytotoxic to murine peritoneal macrophages. (A) Cells were incubated with various concentrations of ZAA for 72 h. Cell viability was determined by colorimetric tetrazolium (MTS) and sulphorhodamine B (SRB) assays. The absorbance was measured at 490 and 590 nm for MTS and SRB assays, respectively. (B) Cells were incubated with various concentrations of ZAA for 1 h, followed by stimulation with LPS (0.5 μg/mL) for 72 h. Cell viability was determined by the SRB assay. Values are means±SD of at least three independent experiments (*p<0.05 vs. untreated cells).

FIG. 7. ZAA cannot inhibit S. choleraesuis replication. S. choleraesuis was cultured in LB broth for 24 or 48 h in the presence of various concentrations of ZAA or ampicillin that served as a positive control. Bacterial growth was assessed by measurement of the absorbance at 600 nm and is expressed as means±SD of at least three independent experiments. (***p<0.001 vs. untreated cells).

### DETAILED DESCRIPTION OF THE INVENTION

### Abbreviations Used in the Following Example are as Follows:

MD-2, Myeloid differentiation factor-2;
ZAA, Zhankuic acid A;
CD14, Cluster of differentiation 14;
iNOS, inducible nitric oxide synthase;
fMLP, N-formyl-methionyl-leucyl-phenylalanine;
PMA, Phorbol-12-myristate-13-acetate; and
ROS, Reactive oxygen species.

### EXAMPLE

### Materials and Methods

### Cells, Bacteria, and Mice

The RAW264.7 murine macrophage cell line and attenuated Salmonella enterica subsp. enterica serovar Choleraesuis (S. choleraesuis) (13) were obtained from the Bioresource Collection and Research Center (Hsinchu, Taiwan). Male C3H/HeJ, C3H/HeN, and C57BL/6 mice (8- to 10-week-old) were obtained from the National Laboratory Animal Center, Taiwan (Taipei, Taiwan).

### Plasmids and Reagents

The NFKB reporter plasmid p-NFκB-Luc was purchased from Promega (Madison, Wis.). The pβ-actin-LacZ plasmid was derived from pFRL2 plasmid (14) by replacing the firefly luciferase expression cassette driven by the CMV promoter with the β-galactosidase expression cassette driven by the β-actin promoter. The pCMV-Luc reporter plasmid was obtained from Addgene (Cambridge, Mass.). The pEGFP-N1 (ΔEGFP) plasmid containing the kanamycin-resistant gene was derived from pEGFP-N1 by deletion of the EGFP coding region. Antibodies against COX2, iNOS, and TLR₄ were purchased from Santa Cruz (Santa Cruz, Calif.). Antibodies against IkBα, ERK, JNK, Akt, and p38, as well as phospho (p)-IkB kinase (IKK) α/β (pIKKα/β), pNF-κBp65, pERK, pJNK, pAkt, and pp38 were obtained from Cell Signaling (Danvers, Mass.).

### Extraction and Isolation of Fungal Compounds

ZAA was isolated from T. camphorates as previously described (10, 15). The compound was dissolved at a concentration of 2 mg/mL in 40% cyclodextrin (Sigma-Aldrich, St. Louis, Mo.) for use as stock solutions, stored at −20° C., and diluted with cell culture medium prior to each experiment. The final concentration of cyclodextrin used in all experiments was below 0.2%.

### Assay of Anti-Inflammatory Molecules

C57BL/6 mice were injected intraperitoneally with 3% thioglycollate, and their peritoneal macrophages were collected 72 h later. Macrophages were cultured in DMEM supplemented with 10% FBS and 50 μg/mL gentamicin at 37° C. in a humidified atmosphere of 5% CO₂. Cells were pretreated with or without ZAA for 1 h and then incubated with LPS (Sigma-Aldrich; 0.5 μg/mL) or IFN-γ (PeproTech, Rocky Hill, N.J.; 50 ng/mL) for 24 h. Cell lysates were subjected to SDS-PAGE for detection of COX2 and iNOS expression. The presence of nitrite (a metabolite of NO) in the culture medium was analyzed by the Griess assay (Sigma-Aldrich), as previously described (16).

### Immunoblot Analysis

Raw264.7 cells were treated with or without various concentrations of ZAA for 1 h, followed by stimulation with LPS (1 μg/mL) for 30 min and homogenization in RIPA lysis buffer (50 mM Tris-HCl, 150 mM NaCl, 2 mM EDTA, pH 8.0, 1 mM Na₃VO₄, 20 μg/mL leupeptin, 20 μg/mL aprotinin, 1 mM PMSF, and 50 mM NaF). Cell lysates were analyzed by immunoblotting with primary antibodies against pIKKα/β, IkBα, pNF-κBp65, pERK, ERK, pJNK, JNK, pAkt, Akt, pp38, p38, and β-actin, followed by appropriate secondary antibodies Immunoreactive protein bands were detected using an enhanced chemiluminescence (ECL) kit (Pierce Biotechnology, Rockford, Ill.). Relative intensities of the protein bands were normalized to that of β-actin and quantified using Image J software (available at http://rsb.info.nih.gov/ij/).

### Reporter Assay

Subconfluent Raw264.7 cells cultured in 24-well plates were cotransfected with p-NFκB-Luc and pβ-actin-LacZ plasmids using the Neon Transfection System (Invitrogen, Carlsbad, Calif.) according to the manufacturer's protocol. Forty-eight hours post-transfection, cells were cultured in serum-free DMEM with or without ZAA (0.5 or 10 μM) for 1 h and then treated with LPS (1 μg/mL) for 24 h. Cell lysates were harvested and their luciferase activities were determined by a dual-light luciferase and β-galactosidase reporter gene assay system (Tropix, Bedford, Mass.). Relative luciferase activity was measured as luciferase activity divided by β-galactosidase activity to normalize transfection efficiency.

### Molecular Docking

Flexible molecular docking was performed using Dock 5.1 software (17). Kollam partial charges were applied to protein models for force field calculation. Energy-optimized three-dimensional coordinates of small molecules were generated by Marvin 5.2.2 (available at http://www.chemaxon.com) and Balloon 0.6 software (18). Additionally, the Gasteiger partial charges were calculated by applying OpenBabel 2.2.3 software (19). The parameters for the Dock program were set to iteratively generate 1000 orientations and 200 conformers in the MD-2 binding pocket. The docked conformers were re-scored and ranked by HotLig to predict the protein-ligand interactions. HotLig is a molecular surface-directed scoring function, which applies the Connolly surface of a protein for evaluation of molecular interactions. First, the Connolly surface of protein was calculated by PscanMS, a tool in the HotLig package, and then the docked ligand conformers were input for analysis of molecular interactions and calculation of binding energy scores. The rendering of figures for molecular modeling was performed using Chimera software (20).

### Native PAGE

For in vitro binding analysis, pre-determined amounts of LPS or ZAA were sonicated for 3 min and incubated with recombinant human MD-2 (R&D, Minneapolis, Minn.; 0.15 μg) or recombinant human TLR_{4/}MD-2 complex (R&D; 1 μg) at 37° C. for 3 h. Samples were subjected to native PAGE, and the levels of TLR₄-associated or free MD-2 were detected by immunoblotting with two anti-MD-2 antibodies, rabbit polyclonal antibody against MD-2 amino acids 110-160 (abcam, Cambridge, Mass.) and mouse monoclonal antibody against MD-2 amino acids 2-160 (abcam). Signals were detected via ECL.

### ELISA for Cytokine Expression

Raw264.7 cells were incubated with or without various concentrations of ZAA for 1 h, followed by treatment with LPS (0.25 or 0.5 μg/mL) or S. choleraesuis [2×10³ colony-forming units (CFU)/well] for 4 and 6 h for detection of TNF-α and IL-6 levels, respectively, in the supernatants by ELISA kits (R&D).

Mouse Models of LPS- or S. choleraesuis-Induced Inflammatory Responses and Diarrhea

Mice were intraperitoneally pretreated with ZAA (20 mg/kg) or the vehicle (0.2% cyclodextrin in normal saline) for 30 min, followed by intraperitoneal injection with LPS (4 mg/kg). After 6 h, the expression levels of cytokines in the plasma were measured by ELISA. Ten hours after LPS treatment, mice were sacrificed, and the organs were resected and fixed in formalin. The lung and kidney were embedded in paraffin, sectioned, and stained with hematoxylin and eosin. Serum samples were collected for determination of blood urea nitrogen (BUN) and serum creatinine levels. The pEGFP-N1 (ΔEGFP)-transformed S. choleraesuis (2×10⁹ CFU/mouse) was orally administered to mice with or without oral pretreatment of ZAA (2 or 10 mg/kg). Serum samples were collected 6 h later for determining TNF-α and IL-6 levels by ELISA. Fecal samples were collected at 24-h intervals until 96 h after S. choleraesuis infection. S. choleraesuis in the feces was quantified by plating serial dilutions of fecal samples on kanamycin-containing agar plates and counting colonies after overnight incubation at 37° C. Mice were monitored daily for symptoms of diarrhea and loss of body weight. Diarrhea was defined according to a diarrhea score of 0-3 (0=normal pellets, 1=slightly loose feces, 2=loose feces, and 3=watery diarrhea) as previously described (21). The experimental protocol adhered to the rules of the Animal Protection Act of

Taiwan and was approved by the Laboratory Animal Care and Use Committee of the National Cheng Kung University.

### LPS-Induced Sepsis Model

C3H/HeN and C3H/HeJ mice were intraperitoneally pretreated with ZAA (2 or 10 mg/kg) or the vehicle. After 30 min, they were administered intraperitoneally with LPS (20 mg/kg). The mice were monitored every 2-4 h until all C3H/HeN mice in the ZAA-untreated and LPS-treated group expired.

### Luciferase-Based Noninvasive Bioluminescence Imaging

C57BL/6 mice were given ZAA (2 mg/kg) or the vehicle orally, followed by oral administration of pCMV-Luc-transformed S. choleraesuis (2×10⁸ CFU/mouse) 30 min later. After 48 h, mice were intraperitoneally injected with D-luciferin potassium salt (Promega, 2.5 mg in 100 μL). They were then anesthetized with 2% isoflurane. In vivo bioluminescence imaging and quantification of signals were performed using the IVIS-200 System and its integrated acquisition and analysis software (Living Image V. 2.50) (Perkin Elmer, Fosty City, Calif.).

### Statistical Analysis

Results are presented as means±standard deviation (SD). Statistical differences were analyzed using Student's unpaired t-test and SigmaPlot™ software (Systat). P values of less than 0.05 were considered statistically significant.

### Results

ZAA Dose-Dependently Inhibits the Production of iNOS, COX2, and NO

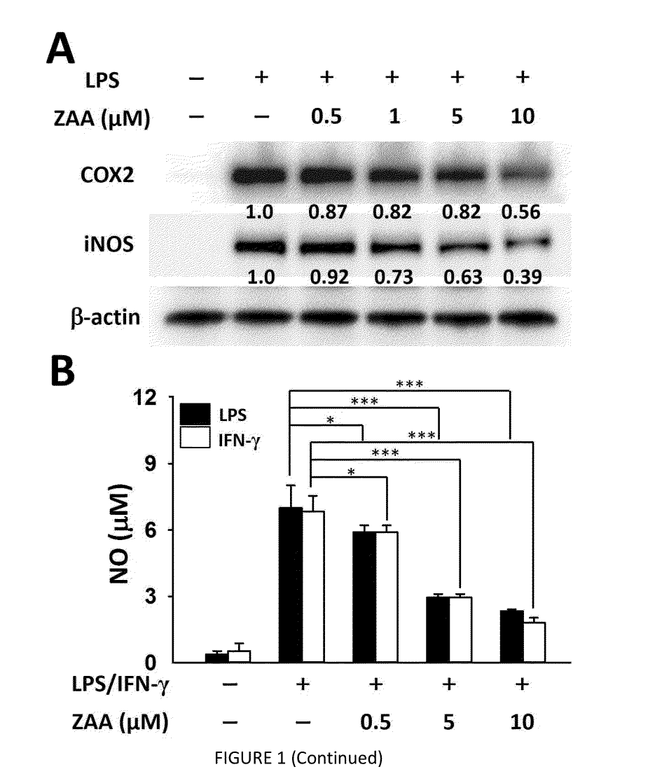

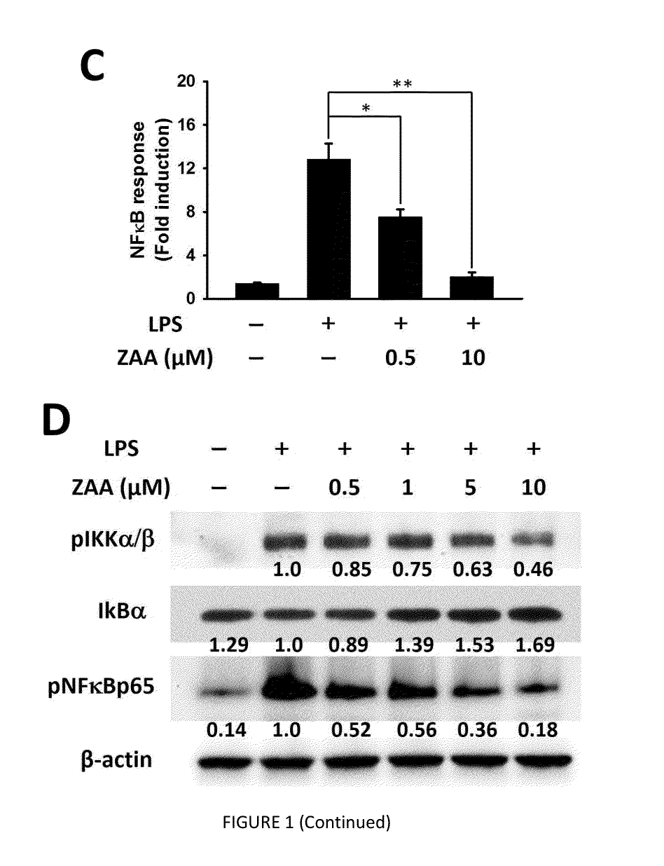

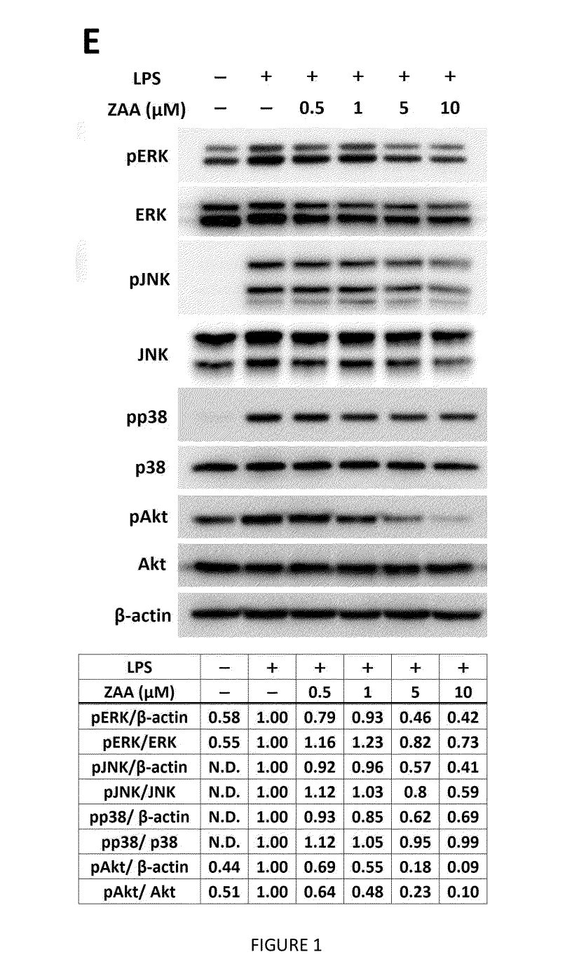

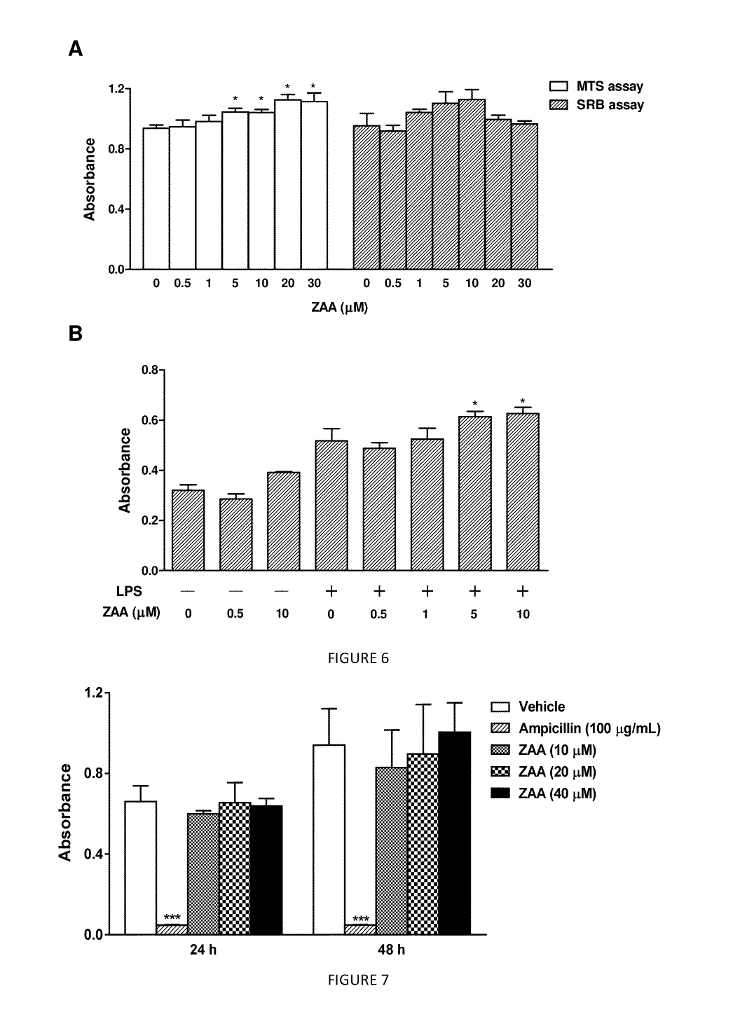

We first investigated the anti-inflammatory properties of ZAA purified from T. camphoratus. During inflammation, large amounts of pro-inflammatory mediators, NO, and prostaglandin E2 (PGE2) are generated by iNOS and COX2. ZAA downregulated the levels of COX2 and iNOS induced by LPS in murine peritoneal macrophages (FIG. 1A), as well as inhibited LPS- and IFN-γ-induced NO production (FIG. 1B) in a dose-dependent manner However, treatment of ZAA up to 30 μM for 72 h did not exert any cytotoxic effects on resident or LPS-activated macrophages, as determined by colorimetric tetrazolium (MTS) and sulphorhodamine B (SRB) assays (22), as shown in FIG. 6.

### ZAA Blocks LPS-Induced NF-κB, MAPK, and Akt Signaling Pathways

To investigate the inhibitory role of ZAA in LPS-stimulated NF-κB signaling, we first detected its effect on the transactivation of NF-κB. FIG. 1C shows that ZAA inhibited NF-κB-mediated transactivation in Raw264.7 cells, as determined by the luciferase reporter assay. Furthermore, LPS treatment stimulated NF-κBp65 phosphorylation, which was significantly prevented by ZAA (FIG. 1D) Similarly, LPS-induced phosphorylation of ERK, JNK, p38, and Akt was also inhibited by ZAA (FIG. 1E). Collectively, these results strongly suggest that ZAA can suppress LPS-stimulated NF-κB, MAPK, and Akt signaling pathways.

ZAA Interacts with the Hydrophobic Pocket of MD-2 to Block LPS Actions

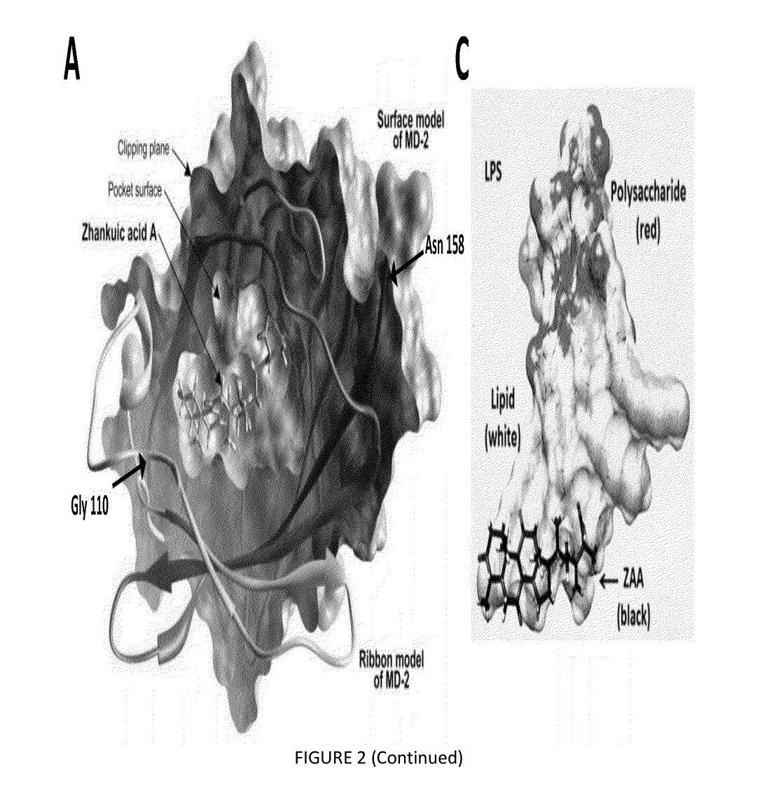

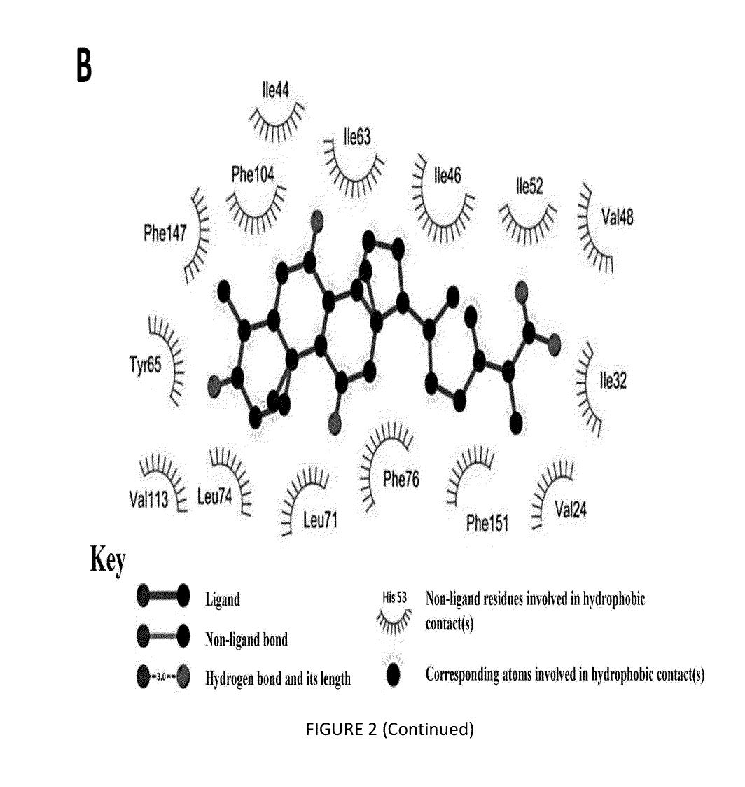

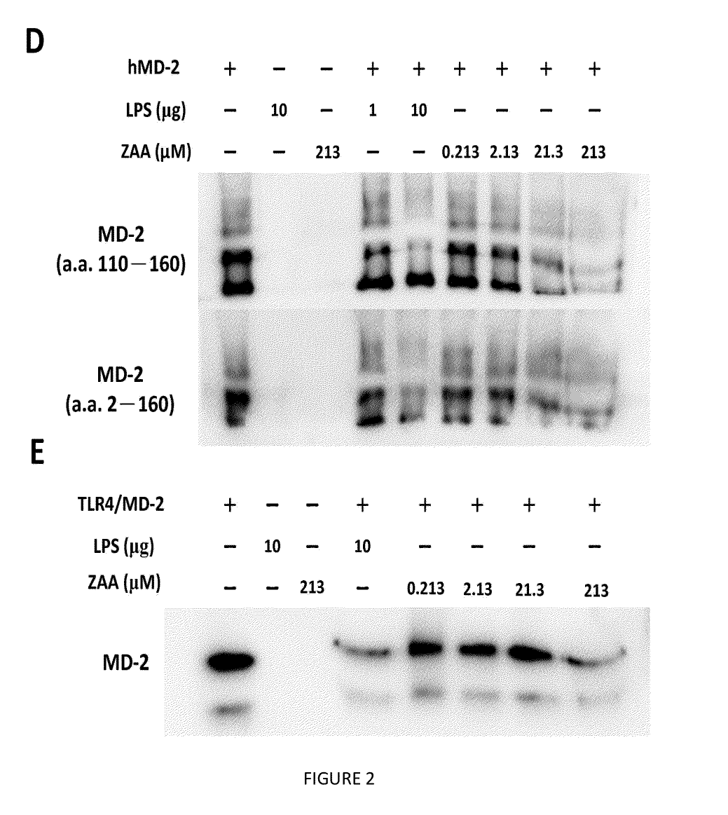

It has been demonstrated that MD-2 in association with the extracellular domain of TLR₄ can trigger LPS-mediated responses (23, 24). To investigate whether ZAA interrupts TLR₄ signaling by competing the binding of LPS to MD-2, we applied an in silico molecular docking analysis to simulate the interactions between ZAA and MD-2. Previous studies have shown that knowledge-based scoring functions are better methods for prediction of protein-ligand interactions, whereas empirical-based scoring functions are more effective for prediction of ligand-binding affinities (25, 26). Therefore, we used a new knowledge-based scoring program, HotLig, to predict the molecular interactions between ZAA and MD-2. Furthermore, we applied an empirical-based scoring program, X-Score, to predict their binding affinities. The HotLig showed about 85%˜90% of success rates for prediction of ligand binding poses (12). On the other hand, the X-Score was reported to be the best scoring function for ranking protein-ligand affinities while comparing with many other well-known scoring programs (25, 26). FIG. 2A presents the clipped surface model of MD-2 (PDB entry: 3FXI) along with the ribbon model to depict the LPS-binding pocket buried within the MD-2 protein. Notably, ZAA assumes a matched configuration to fit into the previously identified LPS-binding pocket. FIG. 2B shows the interactions between ZAA and the hydrophobic amino acid residues of MD-2 (e.g. Ile, Val, Phe, Leu, and Tyr), which constitute the LPS-binding pocket. Clearly, ZAA contacts the pocket by hydrophobic interactions (radiating line-semicircle symbols in FIG. 2B), without hydrogen bonding. Additionally, as shown in FIG. 2C, the molecular superimposition of MD-2-bound ZAA (depicted in black) and LPS (co-crystallized ligand within the 3FXI MD-2 structure) implies that ZAA can occupy the space otherwise occupied by the terminal carbon chains of LPS.

To evaluate the potential binding affinities of ZAA and LPS for MD-2, a consensus scoring analysis was performed using the X-Score scoring functions after generating binding pose predictions via HotLig (Table 1). The predicted pKd (the average of the HPScore, HMScore, and HSScore) of ZAA was 7.83, while that of LPS was 5.83 (Table 2). Therefore, we hypothesize that the matched molecular configuration of ZAA, coupled with the significant hydrophobic interaction effect, can provide a sufficient binding force to stabilize the MD-2/ZAA complex. Thus, ZAA might interfere with LPS/TLR₄ signaling by competing with LPS for binding to MD-2.

TABLE 1

| COMPOUND | Structure | HotLig score |
|---|---|---|
| Zhankuic acid A | 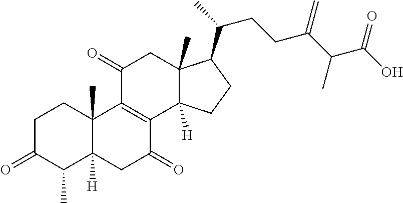 [SMILES: C=C(CC[C@@H](C)[C@H]1CC[C@H]2C3=C(C(=O)C[C@]12C)[C@@]1(C)CCC(=O)[C@@H](C)[C@@H]1CC3=O)C(C)C(=O)O] | −37.895855 |

TABLE 2
Binding affinities of ZAA and LPS as predicted by the X-Score

| Formula | MW | LogP | HPScore | HMScore | HSScore | Average | Name |
|---|---|---|---|---|---|---|---|
| C₂₉H₄₀O₅ | 468.3 | 2.94 | 8.71 | 7.58 | 7.20 | 7.83 | ZAA |
| C₁₃₁H₂₃₆N₂O₅₁ | 2653.3 | 12.21 | 7.42 | 7.44 | 2.63 | 5.83 | LPS |

MW, molecular weight; LogP, partition coefficient.
HPScore = C0, 1 + CVDW, 1*(VDW) + CHB, 1*(H-Bond) + CHP*(Hydrophobic Pair) + CRT, 1*(Rotor)
HMScore = C0, 2 + CVDW, 2*(VDW) + CHB, 2*(H-Bond) + CHM*(Hydrophobic Match) + CRT, 2*(Rotor)
HSScore = C0, 3 + CVDW, 3*(VDW) + CHB, 3*(H-Bond) + CHS*(Hydrophobic Surface) + CRT, 3*(Rotor)
X-Score = (HPScore + HMScore + HSScore)/3

Previous studies have demonstrated that LPS has a similar affinity for MD-2 and the TLR_{4/}MD-2 complex, suggesting that MD-2 is the predominant LPS-binding component (27). Furthermore, the monomeric form of soluble recombinant MD-2 binds LPS, producing a stable MD-2/LPS complex; this complex is sufficient to induce TLR₄-dependent activation (28, 29). We next performed immunoelectrophoresis with two anti-MD-2 antibodies with different antigenic determinants, one polyclonal antibody against MD-2 amino acids 110-160, and the other monoclonal antibody against MD-2 amino acids 2-160. Binding of ZAA to human MD-2 reduced the recognition of both antibodies to native MD-2, similar to the effect of LPS binding (FIG. 2D). A recombinant human TLR_{4/}MD-2 protein complex was then used to measure the binding efficiency of ZAA. FIG. 2E shows that 10 g of ZAA was approximately equivalent to 10 g of LPS. Hence, ZAA might interfere with the recognition of anti-MD-2 antibody to MD-2 through competitive binding with the antibody or causing the conformational change of the MD-2 protein.

ZAA Reduces LPS- and S. choleraesuis-Induced Pro-Inflammatory Cytokine Production

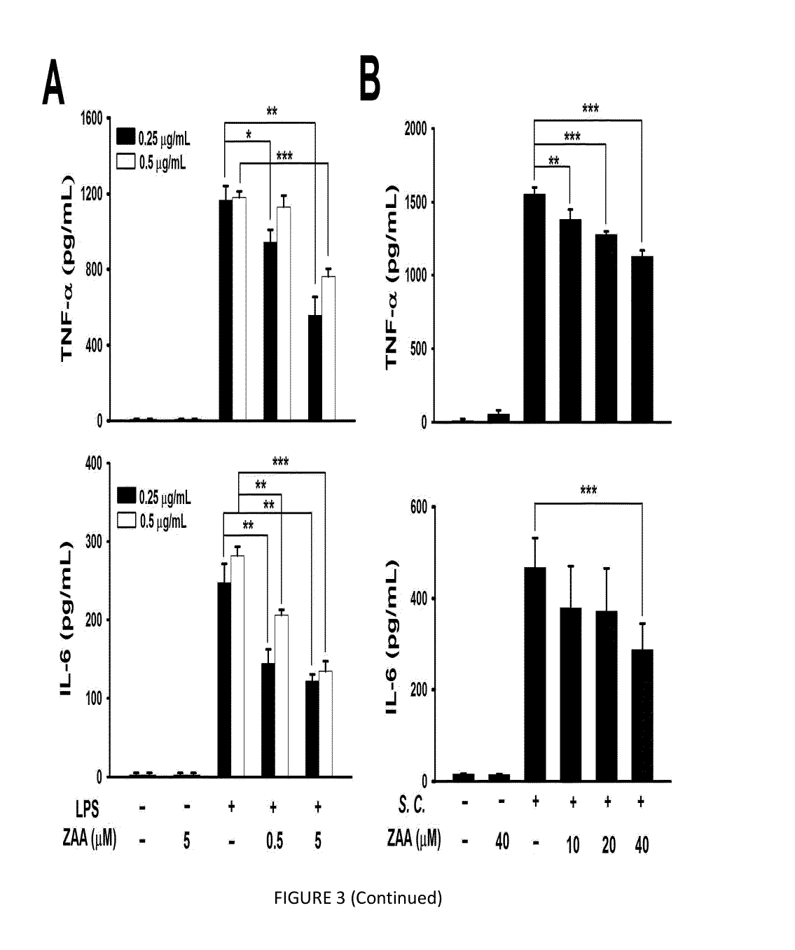

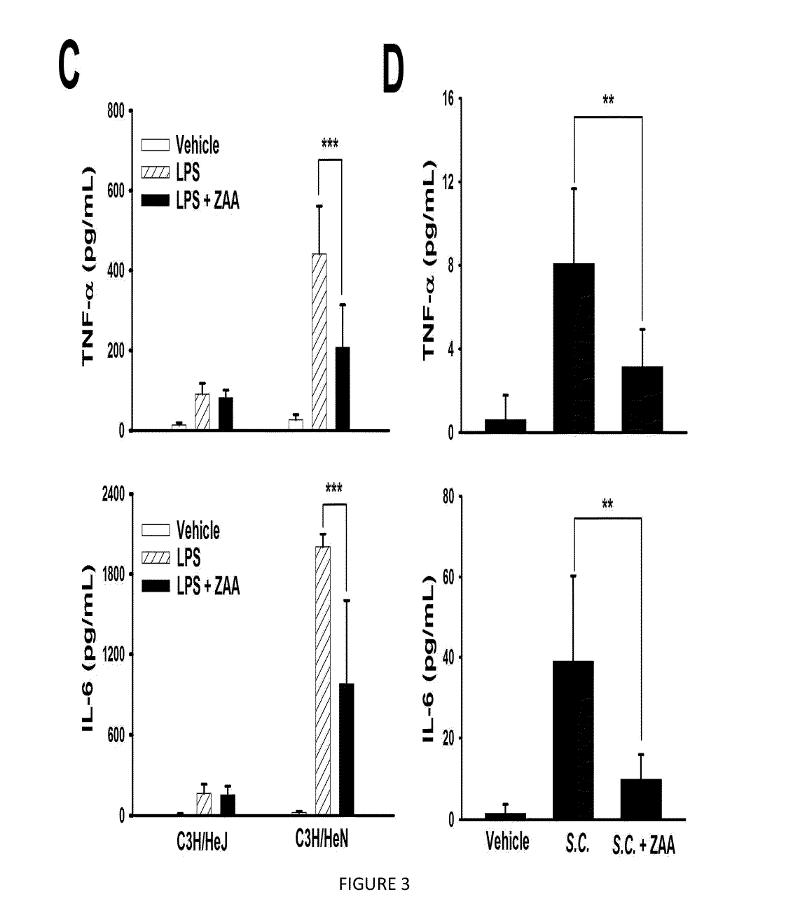

As LPS induces pro-inflammatory cytokine production, we next compared the effects of ZAA on LPS- and S. choleraesuis-induced production of TNF-α and IL-6 in vitro and in vivo. ZAA inhibited LPS-induced TNF-α and IL-6 production in Raw264.7 macrophages at the two concentrations tested (FIG. 3A). To demonstrate whether such effects were also observed in vivo, ZAA was administered to mice 30 min before injection of LPS. While ZAA significantly reduced TNF-α and IL-6 production in C3H/HeN mice, a much weaker response was observed in TLR₄ signaling-defective C3H/HeJ mice (FIG. 3B), suggesting that the actions of ZAA are selective for the LPS/TLR_{4/}MD-2 pathway.

We next explored the effects of ZAA on S. choleraesuis-mediated pro-inflammatory cytokine production both in vitro and in vivo. Our results show that pretreatment of ZAA for 1 h effectively suppressed S. choleraesuis-induced TNF-α and IL-6 production in RAW264.7 cells (FIG. 3C) and C57BL/6 mice (FIG. 3D).

### ZAA Attenuates LPS-Induced Lung and Renal Injury and Lethality

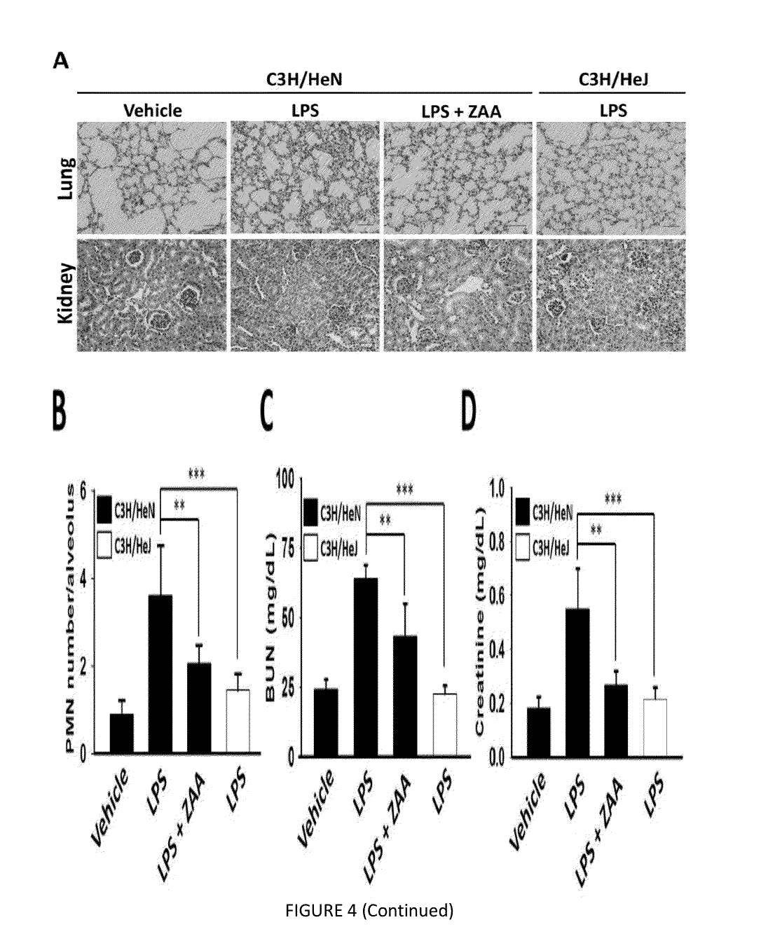

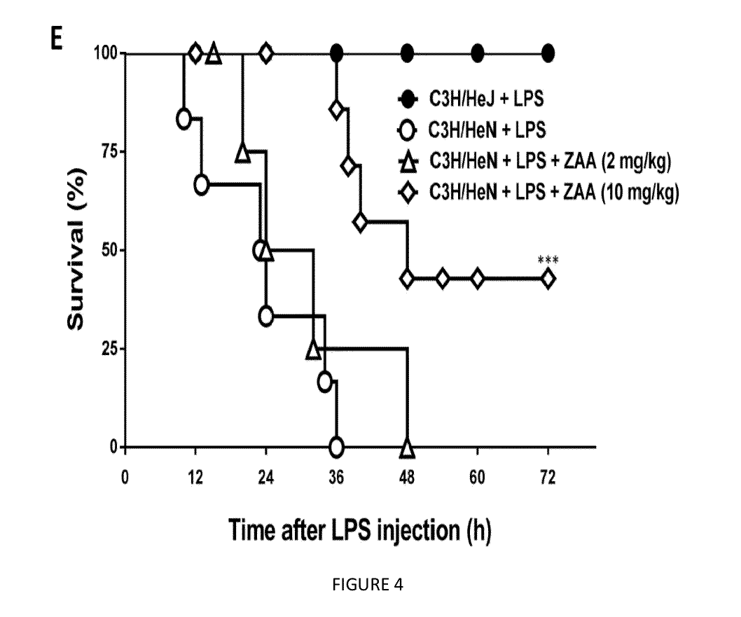

Because ZAA inhibited LPS-induced pro-inflammatory cytokine production and signaling pathways, we then explored ZAA to reduce organ pathology and lethality provoked by LPS in vivo. We determined the inflammation responses in lung and kidney tissues due to their constitutive TLR₄ expression (30, 31). The infiltration of polymorphonuclear leukocytes (PMNs) was elevated in the lung after administration of LPS to C3H/HeN mice; however, ZAA treatment significantly prevented LPS-induced pulmonary accumulation of PMNs (FIGS. 4A and 4 B). Similarly, the extent of LPS-induced glomerulonephritis was significantly reduced in ZAA-pretreated C3H/HeN mice (FIG. 4A). Consistent with the functional failure of the kidney, BUN and serum creatinine levels were increased after LPS administration. ZAA markedly reduced the production of both kidney injury markers (FIGS. 4C and 4D). Notably, LPS treatment did not induce any pathological changes in TLR₄ signaling-defective C3H/HeJ mice (FIGS. 4A-4D).

To evaluate the protective efficacy of ZAA against LPS-induced lethality, C3H/HeN and C3H/HeJ mice were treated with ZAA or the vehicle and challenged with LPS. ZAA significantly protected C3H/HeN mice against lethality and improved survival during endotoxemia (FIG. 4E), suggesting that ZAA may have therapeutic potential against LPS-induced sepsis and gram-negative bacterial infections in general.

ZAA Ameliorates Clinical Symptoms of Mice Infected with S. choleraesuis

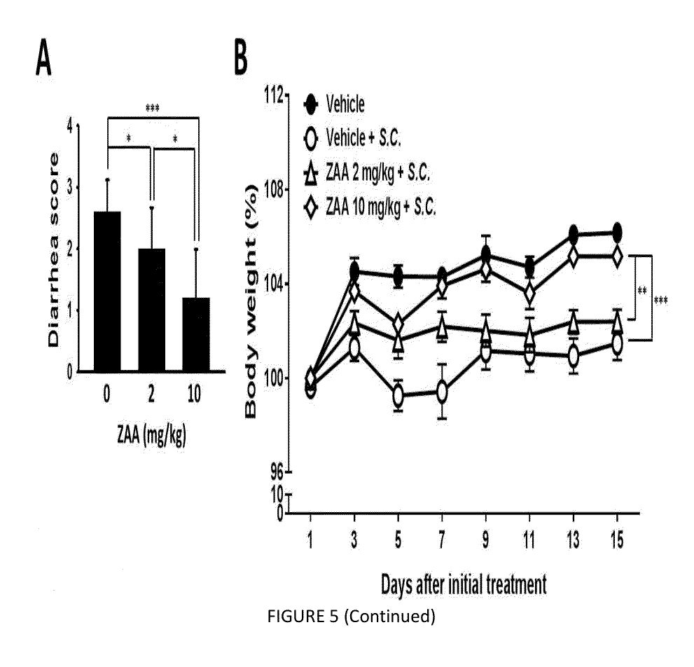

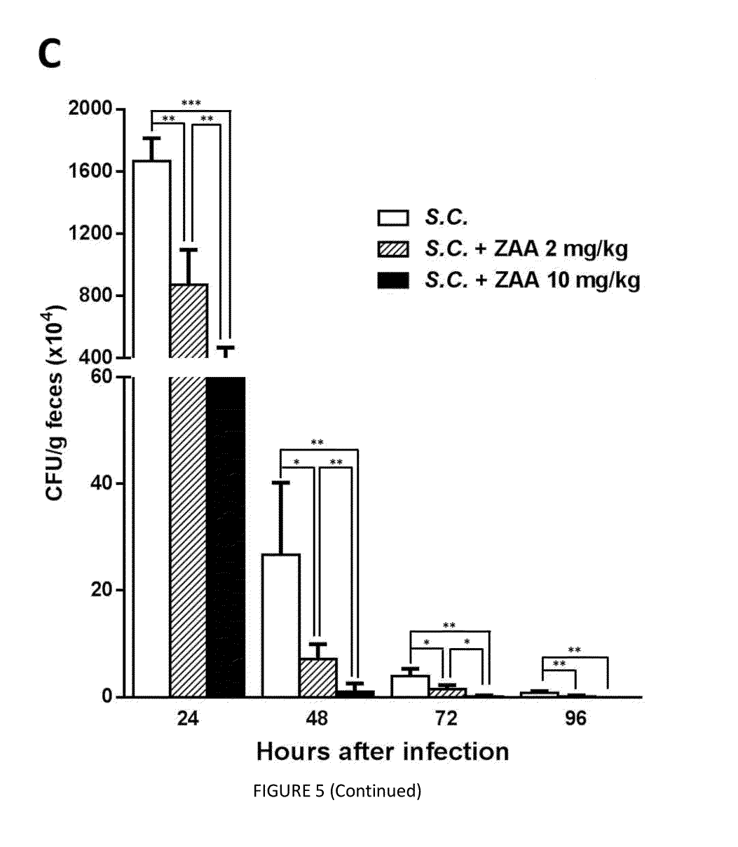

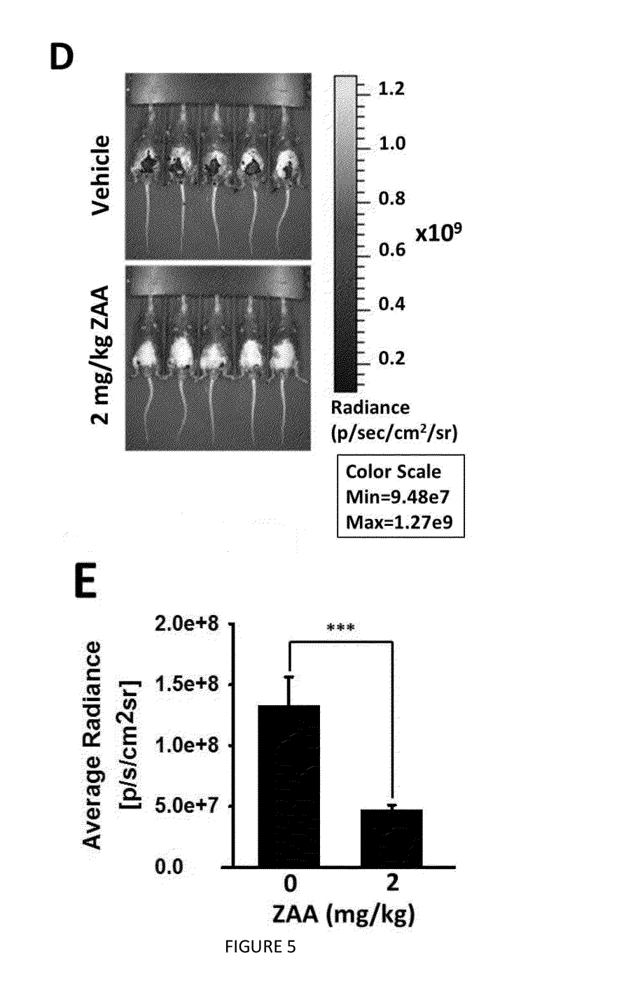

TLR₄ plays a significant role in host defense responses against Salmonella infections (32, 33). Previous studies have shown that mice lacking TLRs, especially TLR_{4,} are more resistant to Salmonella infections (32, 34), suggesting that blockade of LPS on the outer membrane of the bacterium and hence the TLR_{4/}MD-2 interaction is a promising anti-bacterial strategy. To further confirm the anti-inflammatory properties of ZAA in vivo, S. choleraesuis-infected C57BL/6 mice were treated with ZAA, and diarrhea and body weight was monitored for 2 days and 2 weeks, respectively. FIG. 5A shows that ZAA pretreatment reduced the diarrhea score in the infected mice. ZAA treatment (10 mg/kg) dramatically attenuated body weight loss (FIG. 5B), resulting in a return to normal body weight within 2 weeks. As shown in FIG. 5C, the bacterial load in the feces of the untreated mice gradually declined over time. Notably, fecal bacterial load was dramatically reduced in dose- and time-dependent manners in ZAA-treated mice. At 96 h after bacterial infection, fecal bacterial level was undetectable in mice treated with 10 mg/kg of ZAA. Furthermore, mice infected with S. choleraesuis carrying the pCMV-Luc plasmid exhibited much weaker bioluminescence following oral treatment with ZAA than with the vehicle (FIGS. 5D and 5E). Taken together, these results show that ZAA can effectively suppress S. choleraesuis infection and attenuate the clinical symptoms in mice.

### Discussion

In this invention, we used two different modeling approaches and antibody recognition to show for the first time that ZAA interacts with the hydrophobic pocket of MD-2 to block LPS actions. We found that ZAA can act as a ligand for MD-2, thereby suppressing the interaction of LPS with MD-2. We also showed that systemic administration of ZAA protects mice from LPS-induced lung and renal injury and Salmonella-induced enteritis and body weight loss. Our results indicate that ZAA possesses anti-inflammatory activity and may be a potential therapeutic agent for septic shock.

Methanol extracts of the fruiting body of T. camphorates inhibit COX2, iNOS, and TNF-α production in LPS/IFN-γ-activated microglia (8), suggesting that their anti-inflammatory properties might be attributable to the suppression of ERK, JNK and, NF-κB phosphorylation. Given that ZAA is the major pharmacologically active compound in the fruiting body, this invention investigated the anti-inflammatory properties of ZAA. Previous studies have revealed that ZAA inhibits ROS production in fMLP- or PMA-activated peripheral human neutrophils (9) Similarly, we demonstrated that ZAA modulates NO production and attenuates the expression of pro-inflammatory mediators (e.g. iNOS, COX2, TNF-α, and IL-6) in activated murine macrophages.

Antibodies against the TLR_{4/}MD-2 complex have shown efficacy for the treatment of LPS-evoked acute inflammatory conditions (35, 36). Molecules capable of blocking TLR_{4/}MD-2 heterodimer formation and the initiation of inflammation have also been explored recently. For example, eritoran, a synthetic tetraacylated lipid A, competes with LPS for the same binding site in MD-2 and impairs the formation of the LPS-activated receptor complex, which sequentially inhibits signal transmission across the plasma membrane (37). Unfortunately, the phase III study of eritoran showed no significant differences between the treatment and placebo groups (38). Recently, natural and synthetic chemicals that are unrelated to the structure of bacterial lipid A have also been reported to be MD-2-directed LPS antagonists (39). The mechanisms of binding the MD-2 pocket can be divided into three general types: (1) competition for entry into the MD-2 pocket [e.g. bis-ANS (1-anilinonaphthalene 8-sulfonate) and paclitaxel]; (2) covalent interaction with the Cys133 residue within the MD-2 pocket (e.g. N-pyrene maleimide, auranofin, and JTT-705); and (3) linear alignment at the mouth of the bottom interior portion of the pocket (e.g. JSH, curcumin, xanthohumol, and isoxanthohumol). ZAA competes with LPS for entry into the MD-2 pocket, therefore the therapeutic efficacy of ZAA could possibly be improved by increasing its solubility in the blood, or by enhancing its ability to target the MD-2 pocket via LPS-binding protein recognition.

Salmonella are associated with bacteremia, typhoid fever, and enteritis in humans. Most studies on Salmonella pathogenesis have used S. enterica subsp. enterica serovar Typhimurium (S. typhimurium) infection model in mice. While S. typhimurium infection in mice results in a typhoid fever-like disease, this microorganism exclusively causes enteritis in humans (40). Since mice infected with S. typhimurium do not develop diarrhea, the mouse typhoid model is not a good model for investigating enteritis caused by Salmonella infection. On the other hand, S. choleraesuis produces both enteritis and bacteremia in swine, which is its natural host, but also causes diseases in humans (41). Moreover, infection of S. choleraesuis can lead to bacteremia and death in mice (42, 43). Therefore, mice infected with S. choleraesuis can serve as a suitable model of Salmonella-induced bacteremia and enteritis. Since we have used LPS-induced sepsis model to show that pretreatment of ZAA prior to a lethal LPS challenge can improve the survival of mice (FIG. 4E), it is desirable to use S. choleraesuis infection model to further investigate the effects of ZAA on the amelioration of enteritis in mice. In this invention, we used an attenuated strain of S. choleraesuis, which is a vaccine candidate for swine. Infection of mice with this strain leads to diarrhea and body weight loss, making it suitable for investigating the effects of ZAA on Salmonella infection. Our results revealed that ZAA pretreatment ameliorates clinical symptoms of S. choleraesuis-infected mice in terms of the severity of diarrhea, changes in body weight, and fecal bacterial loads.

In conclusion, we show that ZAA effectively ameliorates the inflammatory responses resulting from LPS- or S. choleraesuis-induced experimental endotoxemia, possibly due to its specific interaction with the MD-2 pocket in macrophages. Furthermore, alleviation of inflammation may be attributed to the reduced Salmonella levels in the feces. However, ZAA has no direct inhibitory effects on the growth of Salmonella (FIG. 7). Moreover, our observations strongly support the hypothesis that ZAA can modulate S. choleraesuis-induced diarrhea without provoking adverse effects that are common to many antibiotics (i.e. harmful immune responses attributable to the killing of beneficial bacteria). Collectively, this invention provides novel insights into the mechanisms of ZAA and its analogs as anti-inflammatory agents for LPS-mediated infections in view of above data and the supporting data list in Table 3.

TABLE 3
Anti-inflammatory activity of zhankuic acids in LPS-induced TNF-α
expression*

| | | Inhibition | Inhibition | |
|---|---|---|---|---|
| | | of | of | |
| | | 2 h-TNF-α | 4 h-TNF-α | |
| No | Compound | (%) | (%) | Anti-inflammation |
| 1 | Zhankuic acid A | 42.42 | 85.59 | + |
| 2 | Zhankuic acid A methyl ester | 51.57 | 80.31 | + |
| 3 | Zhankuic acid B | 68.9 | 88.6 | + |
| 4 | Zhankuic acid C | 67.46 | 88.82 | + |
| 5 | Zhankuic acid C 3-O-formate | 1.62 | 45.28 | + |
| 6 | Zhankuic acid D | 67.08 | 54.82 | + |
| 7 | Antcin A | 40.59 | 90.71 | + |
| 8 | Antcin A methyl ester | 19.43 | 85.22 | + |
| 9 | Antcin C | 43.62 | 43.08 | + |
| 10 | Antcin K | 49.39 | 88.45 | + |
| 11 | Antcin M 3-O-formate | — | 88.28 | + |
| 12 | Camphoratin A | 22.76 | 29.91 | + |
| 13 | Camphoratin B | 40.66 | 87.02 | + |
| 14 | Camphoratin D | 65.79 | 81.72 | + |
| 15 | Camphoratin E | 78.02 | 89.39 | + |
| 16 | Camphoratin F | 38.21 | 80.21 | + |
| 17 | Camphoratin G | 68.82 | 22.52 | + |
| 19 | Camphoratin J | 73.43 | 90.25 | + |
| 21 | Camphoratin L | 73.94 | 30.65 | + |
| 22 | Camphoratin N | 58.32 | 88.98 | + |
| 25 | Ergosterol peroxide | — | — | − |
| 26 | Methyl-4α-methylergost-8,24(28)- | 49.32 | — | + |
| | dien-3,11-dion-26-oate | | | |
| 27 | Methyl antcinate H | 79.8 | 91.5% | + |

*Murine peritoneal macrophages were incubated with ZAA analogs at 10 μM for 1 h, followed by stimulation with LPS (1 μg/mL) for 2 h and 4 h to induce TNF-α expression. Supernatant TNF-α was analyzed by ELISA. Inhibitory activities were determined by values compared to positive control (LPS only). Similar results were obtained in at least three independent experiments.

### REFERENCES

1. Bos, M. P., V. Robert, and J. Tommassen. 2007. Biogenesis of the gram-negative bacterial outer membrane. Ann. Rev. Microbiol. 61: 191-214.
2. Bryant, C. E., D. R. Spring, M. Gangloff, and N. J. Gay. 2010. The molecular basis of the host response to lipopolysaccharide. Nat. Rev. Microbiol. 8: 8-14.
3. Song, D. H., and J. O. Lee. 2012. Sensing of microbial molecular patterns by Toll-like receptors. Immunol. Rev. 250: 216-229.
4. Guha, M., and N. Mackman 2001. LPS induction of gene expression in human monocytes. Cell Signal. 13: 85-94.
5. Doyle, S. L., and L. A. O'Neill. 2006. Toll-like receptors: from the discovery of NFKB to new insights into transcriptional regulations in innate immunity. Biochem. Pharmacol. 72: 1102-1113.
6. Rossol, M., H. Heine, U. Meusch, D. Quandt, C. Klein, M. J. Sweet, and S. Hauschildt. 2011. LPS-induced cytokine production in human monocytes and macrophages. Crit. Rev. Immunol. 31: 379-446.
7. Geethangili, M., and Y. M. Tzeng. 2011. Review of pharmacological effects of Antrodia camphorata and its bioactive compounds. Evid. Based Complement. Alternat. Med. Article ID 212641, 17 pages, 2011. doi:10.1093/ecam/nep108
8. Liu, D. Z., H. J. Liang, C. H. Chen, C. H. Su, T. H. Lee, C. T. Huang, W. C. Hou, S. Y. Lin, W. B. Zhong, P. J. Lin, L. F. Hung, and Y. C. Liang. 2007. Comparative anti-inflammatory characterization of wild fruiting body, liquid-state fermentation, and solid-state culture of Taiwanofungus camphorates in microglia and the mechanism of its action. J. Ethnopharmacol. 113: 45-53.
9. Shen, Y. C., Y. H. Wang, Y. C. Chou, C. F. Chen, L. C. Lin, T. T. Chang, J. H. Tien, and C. J. Chou. 2004. Evaluation of the anti-inflammatory activity of zhankuic acids isolated from the fruiting bodies of Antrodia camphorata. Planta Med. 70: 310-314.
10. Wu, S. J., Y. L. Leu, C. H. Chen, C. H. Chao, D. Y. Shen, H. H. Chan, E. J. Lee, T. S. Wu, Y. H. Wang, Y. C. Shen, K. Qian, K. F. Bastow, and K. H. Lee. 2010. Camphoratins A-J, potent cytotoxic and anti-inflammatory triterpenoids from the fruiting body of Taiwanofungus camphorates. J. Nat. Prod. 73: 1756-1762.
11. Wang, R., L. Lai, and S. Wang. 2002. Further development and validation of empirical scoring functions for structure-based binding affinity prediction. J. Comput. Aided. Mol. Des. 16: 11-26.
12. Wang, S. H., Y. T. Wu, S. C. Kuo, and J. Yu. 2013. HotLig: A Molecular Surface-Directed Approach to Scoring Protein-Ligand Interactions. J. Chem. Inf. Model. 53: 2181-2195.
13. Lee, C. H., C. L. Wu, and A. L. Shiau. 2008. Toll-like receptor 4 mediates an antitumor host response induced by Salmonella choleraesuis. Clin. Cancer Res. 14: 1905-1912.
14. Malo, M. S., M. Abedrapo, A. Chen, M. Mozumder, P. Pushpakaran, F. Alkhoury, W. Zhang, E. Fleming, and R. A. Hodin. 2003 Improved eukaryotic promoter-detection vector carrying two luciferase reporter genes. Biotechniques 35: 1150-1152, 1154.
15. Shi, L. S., C. H. Chao, D. Y. Shen, H. H. Chan, C. H. Chen, Y. R. Liao, S. J. Wu, Y. L. Leu, Y. C. Shen, Y. H. Kuo, E. J. Lee, K. Qian, T. S. Wu, and K. H. Lee. 2011. Biologically active constituents from the fruiting body of Taiwanofungus camphorates. Bioorg. Med. Chem. 19: 677-683.
16. Fang, J., and R. B. Silverman. 2009. A cellular model for screening neuronal nitric oxide synthase inhibitors. Anal. Biochem. 390: 74-78.
17. Moustakas, D. T., P. T. Lang, S. Pegg, E. Pettersen, I. D. Kuntz, N. Brooijmans, and R. C. Rizzo. 2006. Development and validation of a modular, extensible docking program: DOCK 5. J. Comput. Aided. Mol. Des. 20: 601-619.
18. Vainio, M. J., and M. S. Johnson. 2007. Generating conformer ensembles using a multiobjective genetic algorithm. J. Chem. Inf. Model. 47: 2462-2474.
19. Guha, R., M. T. Howard, G. R. Hutchison, P. Murray-Rust, H. Rzepa, C. Steinbeck, J. Wegner, and E. L. Willighagen. 2006. The Blue Obelisk-interoperability in chemical informatics. J. Chem. Inf. Model. 46: 991-998.
20. Pettersen, E. F., T. D. Goddard, C. C. Huang, G. S. Couch, D. M. Greenblatt, E. C. Meng, and T. E. Ferrin. 2004. UCSF Chimera—a visualization system for exploratory research and analysis. J. Comput. Chem. 25: 1605-1612.
21. Melgar, S., A. Karlsson, and E. Michaëlsson 2005. Acute colitis induced by dextran sulfate sodium progresses to chronicity in C57BL/6 but not in BALB/c mice: correlation between symptoms and inflammation. Am. J. Physiol. Gastrointest. Liver Physiol. 288: G1328-1338.
22. Vichai, V., and K. Kirtikara. 2006. Sulforhodamine B colorimetric assay for cytotoxicity screening Nat. Protoc. 1: 1112-1116.
23. Gioannini, T. L., A. Teghanemt, D. Zhang, E. N. Levis, and J. P. Weiss. 2005. Monomeric endotoxin:protein complexes are essential for TLR₄-dependent cell activation. J. Endotoxin Res. 11: 117-123.
24. Visintin, A., K. A. Halmen, E. Latz, B. G. Monks, and D. T. Golenbock. 2005. Pharmacological inhibition of endotoxin responses is achieved by targeting the TLR₄ coreceptor, MD-2. J. Immunol. 175: 6465-6472.
25. Wang, R., Y. Lu, and S. Wang. 2003. Comparative evaluation of 11 scoring functions for molecular docking J. Med. Chem. 46: 2287-2303.
26. Cheng, T., X. Li, Y. Li, Z. Liu, and R. Wang. 2009. Comparative assessment of scoring functions on a diverse test set. J. Chem. Inf. Model. 49: 1079-1093.
27. Viriyakosol, S., P. S. Tobias, R. L. Kitchens, and T. N. Kirkland. 2001. MD-2 binds to bacterial lipopolysaccharide. J. Biol. Chem. 276: 38044-38051.
28. Gioannini, T. L., A. Teghanemt, D. Zhang, N. P. Coussens, W. Dockstader, S. Ramaswamy, and J. P. Weiss. 2004. Isolation of an endotoxin-MD-2 complex that produces Toll-like receptor 4-dependent cell activation at picomolar concentrations. Proc. Natl. Acad. Sci. USA 101: 4186-4191.
29. Re, F., and J. L. Strominger. 2002. Monomeric recombinant MD-2 binds toll-like receptor 4 tightly and confers lipopolysaccharide responsiveness. J. Biol. Chem. 277: 23427-23432.
30. Guillot, L., S. Medjane, K. Le-Barillec, V. Balloy, C. Danel, M. Chignard, and M. Si-Tahar. 2004. Response of human pulmonary epithelial cells to lipopolysaccharide involves Toll-like receptor 4 (TLR₄)-dependent signaling pathways: evidence for an intracellular compartmentalization of TLR_{4.} J. Biol. Chem. 279: 2712-2718.
31. Vandewalle, A. 2008. Toll-like receptors and renal bacterial infections. Chang Gung Med. J. 31: 525-537.
32. Arpaia, N., J. Godec, L. Lau, K. E. Sivick, L. M. McLaughlin, M. B. Jones, T. Dracheva, S. N. Peterson, D. M. Monack, and G. M. Barton. 2011. TLR signaling is required for Salmonella typhimurium virulence. Cell 144: 675-688.
33. Talbot, S., S. Tötemeyer, M. Yamamoto, S. Akira, K. Hughes, D. Gray, T. Barr, P. Mastroeni, D. J. Maskell, and C. E. Bryant. 2009. Toll-like receptor 4 signalling through MyD88 is essential to control Salmonella enterica serovar typhimurium infection, but not for the initiation of bacterial clearance. Immunology 128: 472-483.
34. Weiss, D. S., B. Raupach, K. Takeda, S. Akira, and A. Zychlinsky. 2004. Toll-like receptors are temporally involved in host defense. J. Immunol. 172: 4463-4469.
35. Daubeuf, B., J. Mathison, S. Spiller, S. Hugues, S. Herren, W. Ferlin, M. Kosco-Vilbois, H. Wagner, C. J. Kirschning, R. Ulevitch, and G Elson. 2007. TLR_{4/}MD-2 monoclonal antibody therapy affords protection in experimental models of septic shock. J. Immunol. 179: 6107-6114.
36. Akashi-Takamura, S., T. Furuta, K. Takahashi, N. Tanimura, Y. Kusumoto, T. Kobayashi, S. Saitoh, Y. Adachi, T. Doi, and K. Miyake. 2006. Agonistic antibody to TLR_{4/}MD-2 protects mice from acute lethal hepatitis induced by TNF-α. J. Immunol. 176: 4244-4251.
37. Barochia, A., S. Solomon, X. Cui, C. Natanson, and P. Q. Eichacker. 2011. Eritoran tetrasodium (E5564) treatment for sepsis: review of preclinical and clinical studies. Expert Opin. Drug Metab. Toxicol. 7: 479-494.
38. Opal, S. M., P. F. Laterre, B. Francois, S. P. LaRosa, D. C. Angus, J. P. Mira, X. Wittebole, T. Dugernier, D. Perrotin, M. Tidswell, L. Jauregui, K. Krell, J. Pachl, T. Takahashi, C. Peckelsen, E. Cordasco, C. S. Chang, S. Oeyen, N. Aikawa, T. Maruyama, R. Schein, A. C. Kalil, M. Van Nuffelen, M. Lynn, D. P. Rossignol, J. Gogate, M. B. Roberts, J. L. Wheeler, and J. L. Vincent. 2013. Effect of eritoran, an antagonist of MD2-TLR_{4,} on mortality in patients with severe sepsis: the ACCESS randomized trial. JAMA 309: 1154-1162.
39. Park, S. H., N. D. Kim, J. K. Jung, C. K. Lee, S. B. Han, and Y. Kim. 2012. Myeloid differentiation 2 as a therapeutic target of inflammatory disorders. Pharmacol. Ther 133: 291-298.
40. Hapfelmeier, S., and W. D. Hardt. 2005. A mouse model for S. typhimurium-induced enterocolitis. Trends Microbiol. 13: 497-503.
41. Chiu, C. H., L. H. Su, and C. Chu. 2004. Salmonella enterica serotype Choleraesuis: epidemiology, pathogenesis, clinical disease, and treatment. Clin. Microbiol. Rev. 17: 311-322.
42. Kawahara, K., Y. Haraguchi, M. Tsuchimoto, N. Terakado, and H. Danbara. 1988. Evidence of correlation between 50-kilobase plasmid of Salmonella choleraesuis and its virulence. Microb. Pathog. 4: 155-163.
43. Emoto, M., H. Nishimura, T. Sakai, K. Hiromatsu, H. Gomi, S. Itohara, and Y. Yoshikai. 1995. Mice deficient in γδ T cells are resistant to lethal infection with Salmonella choleraesuis. Infect. Immun. 63: 3736-3738.

1. A method of treating an inflammatory disease in a subject comprising administering to the subject a compound having the following chemical formula (I) or a pharmaceutically acceptable salt thereof in need of said treatment: 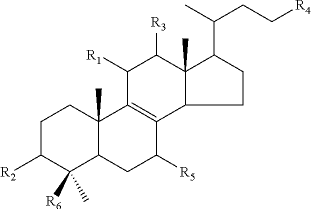
wherein R₁ is ═O; R₂ is ═O, OCHO, or OH; R₃ is H or OH; R₄ is —C(═CH₂)—C(CH₃)H—(C═O)ORₐ, in which Rₐ is H or C₁₋₄ alkyl, or R₄ is —(C═O)R_{b}, in which R_{b} is C₁₋₄ alkyl; R₅ is ═O, OH or H; and R₆ is H or OH.

2. The method of claim 1, wherein the inflammatory disease is a LPS-mediated inflammatory condition.

3. The method of claim 1, wherein the inflammatory disease is caused by bacterial infection.

4. The method of claim 3, wherein the bacterial infection is caused by Gram-negative bacteria.

5. The method of claim 1, wherein the inflammatory disease comprises LPS-induced lung injury or LPS-induced renal injury.

6. The method of claim 1, wherein the inflammatory disease comprises diarrhea.

7. The method of claim 1, wherein the inflammatory disease comprises enteritis.

8. The method of claim 1, wherein the compound having the formula (I) is Zhankuic acid A.

9. The method of claim 1, wherein the compound having the formula (I) is selected from the group consisting of

| No. | Compound | Structure |
|---|---|---|
| 2 | Zhankuic acid A methyl ester | 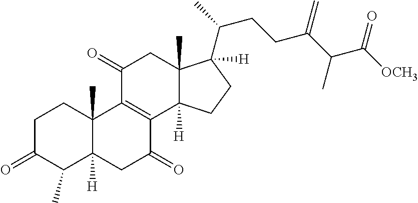 [SMILES: C=C(CC[C@@H](C)[C@H]1CC[C@H]2C3=C(C(=O)C[C@]12C)[C@@]1(C)CCC(=O)[C@@H](C)[C@@H]1CC3=O)C(C)C(=O)OC] |
| 3 | Zhankuic acid B | 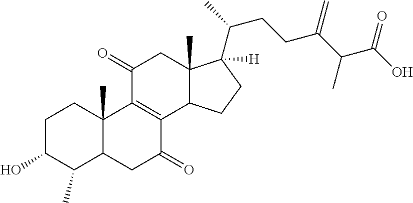 [SMILES: C=C(CC[C@@H](C)[C@H]1CCC2C3=C(C(=O)C[C@@]21C)[C@@]1(C)CC[C@@H](O)[C@@H](C)C1CC3=O)C(C)C(=O)O] |
| 4 | Zhankuic acid C | 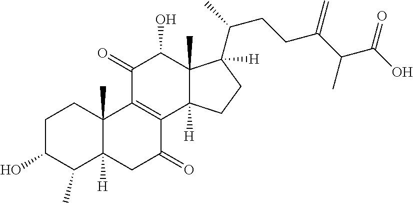 [SMILES: C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)C(C)C(=O)O] |
| 5 | Zhankuic acid C 3-O-formate | 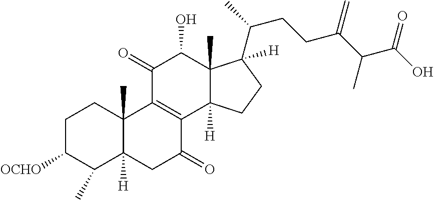 |
| 6 | Zhankuic acid D | 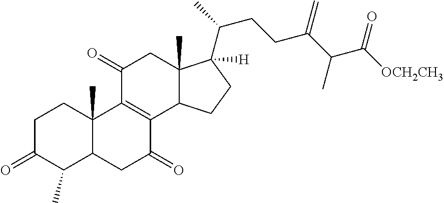 [SMILES: C=C(CC[C@@H](C)[C@H]1CCC2C3=C(C(=O)C[C@@]21C)[C@@]1(C)CCC(=O)[C@@H](C)C1CC3=O)C(C)C(=O)OCC] |
| 7 | Antcin A | 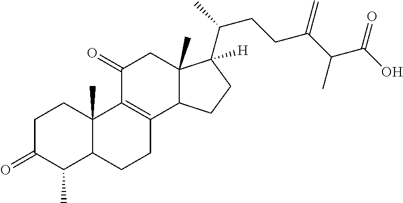 [SMILES: C=C(CC[C@@H](C)[C@H]1CCC2C3=C(C(=O)C[C@@]21C)[C@@]1(C)CCC(=O)[C@@H](C)C1CC3)C(C)C(=O)O] |
| 8 | Antcin A methyl ester | 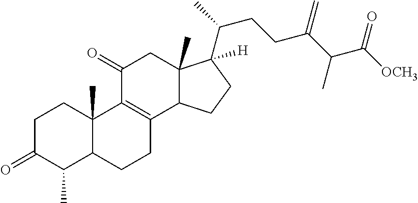 [SMILES: C=C(CC[C@@H](C)[C@H]1CCC2C3=C(C(=O)C[C@@]21C)[C@@]1(C)CCC(=O)[C@@H](C)C1CC3)C(C)C(=O)OC] |
| 9 | Antcin C | 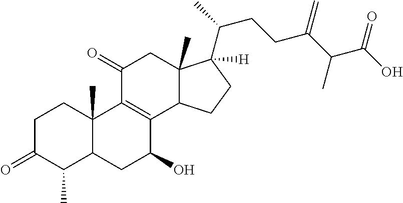 [SMILES: C=C(CC[C@@H](C)[C@H]1CCC2C3=C(C(=O)C[C@@]21C)[C@@]1(C)CCC(=O)[C@@H](C)C1C[C@@H]3O)C(C)C(=O)O] |
| 10 | Antcin K | 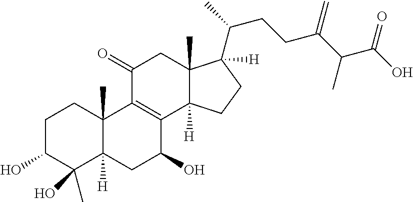 [SMILES: C=C(CC[C@@H](C)[C@H]1CC[C@H]2C3=C(C(=O)C[C@]12C)[C@@]1(C)CC[C@@H](O)[C@](C)(O)[C@@H]1C[C@@H]3O)C(C)C(=O)O] |
| 11 | Antcin M 3-O-formate | 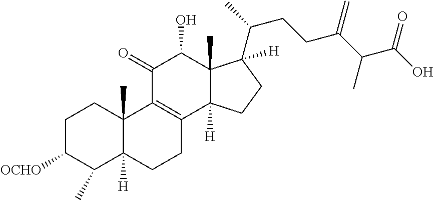 |
| 12 | Camphoratin A | 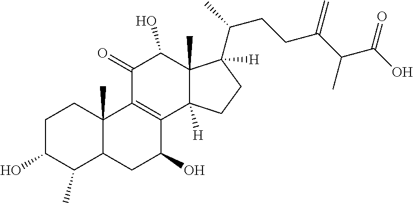 [SMILES: C=C(CC[C@@H](C)[C@H]1CC[C@H]2C3=C(C(=O)[C@H](O)[C@]12C)[C@@]1(C)CC[C@@H](O)[C@@H](C)C1C[C@@H]3O)C(C)C(=O)O] |
| 13 | Camphoratin B | 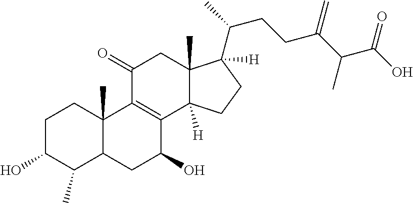 [SMILES: C=C(CC[C@@H](C)[C@H]1CC[C@H]2C3=C(C(=O)C[C@]12C)[C@@]1(C)CC[C@@H](O)[C@@H](C)C1C[C@@H]3O)C(C)C(=O)O] |
| 14 | Camphoratin D | 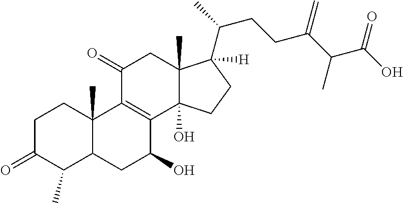 [SMILES: C=C(CC[C@@H](C)[C@H]1CC[C@@]2(O)C3=C(C(=O)C[C@]12C)[C@@]1(C)CCC(=O)[C@@H](C)C1C[C@@H]3O)C(C)C(=O)O] |
| 15 | Camphoratin E | 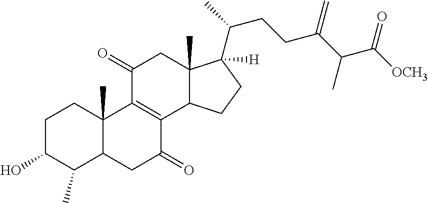 [SMILES: C=C(CC[C@@H](C)[C@H]1CCC2C3=C(C(=O)C[C@@]21C)[C@@]1(C)CC[C@@H](O)[C@@H](C)C1CC3=O)C(C)C(=O)OC] |
| 16 | Camphoratin F | 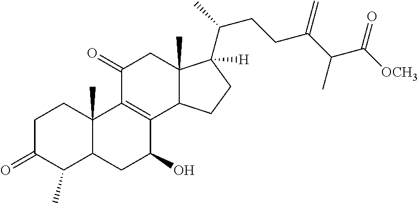 [SMILES: C=C(CC[C@@H](C)[C@H]1CCC2C3=C(C(=O)C[C@@]21C)[C@@]1(C)CCC(=O)[C@@H](C)C1C[C@@H]3O)C(C)C(=O)OC] |
| 17 | Camphoratin G | 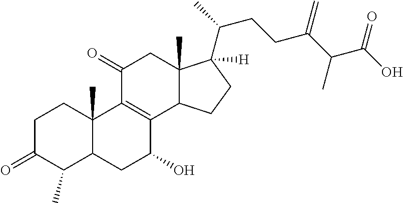 [SMILES: C=C(CC[C@@H](C)[C@H]1CCC2C3=C(C(=O)C[C@@]21C)[C@@]1(C)CCC(=O)[C@@H](C)C1C[C@H]3O)C(C)C(=O)O] |
| 19 | Camphoratin J | 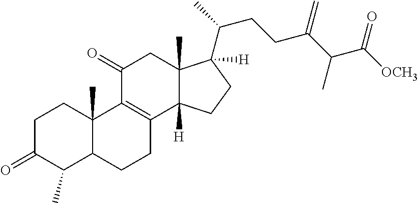 [SMILES: C=C(CC[C@@H](C)[C@H]1CC[C@@H]2C3=C(C(=O)C[C@@]21C)[C@@]1(C)CCC(=O)[C@@H](C)C1CC3)C(C)C(=O)OC] |
| 21 | Camphoratin L | 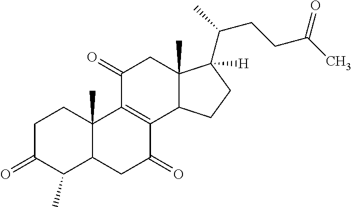 [SMILES: CC(=O)CC[C@@H](C)[C@H]1CCC2C3=C(C(=O)C[C@@]21C)[C@@]1(C)CCC(=O)[C@@H](C)C1CC3=O] |
| 22 | Camphoratin N | 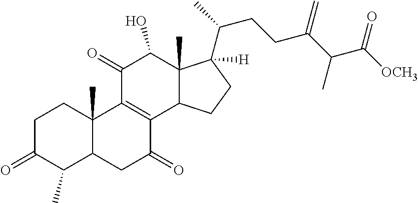 [SMILES: C=C(CC[C@@H](C)[C@H]1CCC2C3=C(C(=O)[C@H](O)[C@@]21C)[C@@]1(C)CCC(=O)[C@@H](C)C1CC3=O)C(C)C(=O)OC] |
| 25 | Ergosterol peroxide | 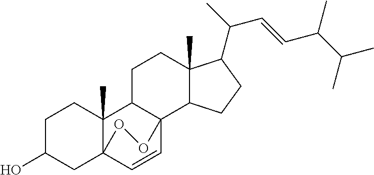 [SMILES: CC(C)C(C)/C=C/C(C)C1CCC2C34C=CC5(CC(O)CC[C@]5(C)C3CC[C@]12C)OO4] |
| 26 | Methyl-4α-methylergost- 8,24(28)-diene-3,11-dion-26- oate | 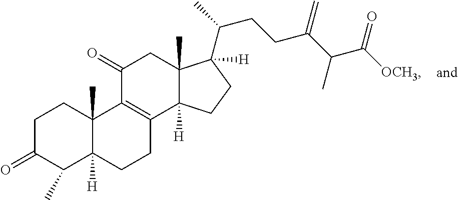 [SMILES: C=C(CC[C@@H](C)[C@H]1CC[C@H]2C3=C(C(=O)C[C@]12C)[C@@]1(C)CCC(=O)[C@@H](C)[C@@H]1CC3)C(C)C(=O)OC ; "and"] |
| 27 | Methyl antcinate H | 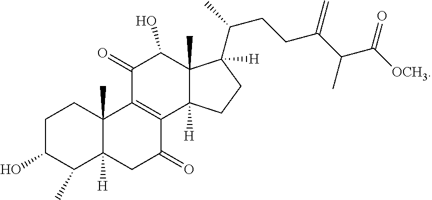 [SMILES: C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)C(C)C(=O)OC] |
